(19) United States
(12) Patent Application Publication
(10) Pub. No.: US 2009/0312414 A1
(43) Pub. Date: Dec. 17, 2009

(54) Treatment Of Lower Urinary Tract Dysfunction With CB2-Receptor-Selective Agonists

(75) Inventors: Jan Susan Rosenbaum, Cincinnati, OH (US); Karl-Erik Andersson, Winston Salem, NC (US); Iris Alroy, Nes Ziona (IL)

Correspondence Address: THE PROCTER & GAMBLE COMPANY;Global Legal Department - IP, Sycamore Building - 4th Floor, 299 East Sixth Street, CINCINNATI, OH, 45202, US

(73) Assignee: The Procter & Gamble Company, Cincinnati, OH (US)

(21) Appl. No.: 12/465,762

(22) Filed: May 14, 2009

Related U.S. Application Data
(60) Provisional application No. 61/071,775, filed on May 16, 2008.

(51) Int. Cl. A61K 31/343; A61K 31/225; A61K 31/047; A61K 31/18; A61P 13/10; A61P 13/02

(52) U.S. Cl. 514/468; 514/548; 514/729; 514/603

(57) ABSTRACT

A method is disclosed utilizing a cannabinoid receptor type 2-receptor-selective agonist for treating or preventing lower urinary tract dysfunction, including overactive bladder, lower urinary tract symptoms and detrusor overactivity.

### CROSS REFERENCE TO RELATED APPLICATIONS

This applications claims priority to U.S. Provisional Application No. 61/071,775, filed May 16, 2008.

### BACKGROUND OF THE INVENTION

The incidence of overactive bladder, a syndrome of lower urinary tract dysfunction, is similar in men and women, but the characteristics of the disease differ with gender, with a larger proportion of women experiencing urge incontinence (overactive bladder wet) than that of men. Overactive bladder affects approximately 12-17% of women in the United States and Europe. (Debra E. Irwin, Ian Milsom, Steinar Hunskaar, Kate Reilly, Zoe Kopp, Sender Herschom, Karin Coyne, Con Kelleher, Christian Hampel, Walter Artibani, and Paul Abrams, “Population-Based Survey of Urinary Incontinence, Overactive Bladder, and Other Lower Urinary Tract Symptoms in Five Countries: Results of the EPIC Study” European Urology 50 (6): 1306-1315 2006; I. Milsom, P. Abrams, L. Cardozo, R. G. Roberts, J. Thuroff, and A. J. Wein. How widespread are the symptoms of an overactive bladder and how are they managed? A population-based prevalence study. BJU. Int. 87 (9):760-766, 2001; W. F. Stewart, J. B. Van Rooyen, G. W. Cundiff, P. Abrams, A. R. Herzog, R. Corey, T. L. Hunt, and A. J. Wein. Prevalence and burden of overactive bladder in the United States. World J Urol. 20 (6):327-336, 2003.) In both men and women, the prevalance of overactive bladder increases substantially with age, with etiology being myogenic, neurogenic or idiopathic in nature. (Chu F M, Dmochowski R. Pathophysiology of Overactive Bladder. The American Journal of Medicine. 2006; 119:3-8.) The mechanism of the control of the micturition response involves receptors located within both the central and peripheral nervous systems (de Groat W C. Integrative control of the lower urinary tract: preclinical perspective. Br J Pharmacol. 2006; 147 Suppl 2:S25-S40; Andersson K E. Mechanisms of Disease: central nervous system involvement in overactive bladder syndrome. Nat Clin Pract Urol. 2004; 1:103-108), and the overactive bladder symptoms seen in patients with spinal cord injury and various neurological disorders such as multiple sclerosis are identical to those with idiopathic disease (Andersson K E. Mechanisms of Disease: central nervous system involvement in overactive bladder syndrome. Nat Clin Pract Urol. 2004; 1: 103-108).

Current drugs used in individuals with lower urinary tract dysfunction mainly include antimuscarinics. Antimuscarinics act on the muscarinic acetylcholine receptors. However, antimuscarinics cause adverse side effects and have limited efficacy. In particular, after six months of treatment with antimuscarinics, ˜80% of patients no longer continue the treatment because the efficacy is not sufficient to outweigh the significant side effect profile. (Kelleher, C. J, Cardozo, L. D., Khullar, V, Salvatore, S. A medium-term analysis of the subjective efficacy of treatment for women with detrusor instability and low bladder compliance. Br J Obstet Gyneacol. 1997; 104:988-993.)

Accordingly, it would be particularly desirable to find efficacious methods of treatment of lower urinary tract dysfunction without the aforementioned adverse effects.

### BRIEF SUMMARY OF THE INVENTION

It has now been discovered that CB2-receptor-selective agonists are effective compounds that are useful for treating or preventing lower urinary tract dysfunction.

The present invention involves treating or preventing lower urinary tract dysfunction in mammals comprising administering a therapeutically effective amount of a CB2-receptor-selective agonist. The invention further relates to treating or preventing overactive bladder, lower urinary tract symptoms or detrusor overactivity with a CB2-receptor-selective agonist.

### BRIEF DESCRIPTION OF THE DRAWINGS

FIG. 1 shows the effect of the compounds of Formulas II, III and IV on threshold pressure (“ThP”) in an acetic acid model.

FIG. 2 shows the effect of the compound of Formula I on micturition interval (“MI”) in conscious normal rats.

FIG. 3 shows the effect of the compound of Formula I on threshold pressure (“ThP”) in conscious normal rats.

FIG. 4 shows the effect of the compound of Formula I on maximal pressure (“MP”) in conscious normal rats.

FIG. 5 shows the effect of the compound of Formula I on micturition volume (“MV”) in conscious normal rats.

FIG. 6 shows the effect of the compound of Formula I on bladder capacity (“BC”) in conscious normal rats.

FIG. 7 shows the effect of the compound of Formula I on MI in conscious rats with partial urethral obstruction (“PUO”).

FIG. 8 shows the effect of the compound of Formula I on micturition frequency (“MF”) in conscious rats with partial urethral obstruction.

FIG. 9 shows the effect of the compound of Formula I on ThP in conscious rats with partial urethral obstruction.

FIG. 10 shows the effect of the compound of Formula I on flow pressure (“FP”) in conscious rats with partial urethral obstruction.

FIG. 11 shows the effect of the compound of Formula I on MP in conscious rats with partial urethral obstruction.

FIG. 12 shows the effect of the compound of Formula I on MV in conscious rats with partial urethral obstruction.

FIG. 13 shows the effect of the compound of Formula I on residual volume (“RV”) in conscious rats with partial urethral obstruction.

FIG. 14 shows the effect of the compound of Formula I on non-voiding contractions (“NVCs”) in conscious rats with partial urethral obstruction.

FIG. 15 shows the effect of the compound of Formula I on carbachol-induced contractions in isolated detrusor from rats with partial urethral obstruction.

FIG. 16 shows the effect of the compound of Formula I on nerve-induced contractions in response to electrical field stimulation (“EFS”) in isolated detrusor from rats with partial urethral obstruction.

FIG. 17 shows the effect of the compound of Formula I on bladder capacity (“BC”) in conscious rats treated with cyclophosphamide.

### DETAILED DESCRIPTION OF THE INVENTION

The present invention encompasses a method of treating or preventing lower urinary tract dysfunction in mammals, which comprises administering a therapeutically effective amount of a cannabinoid receptor type 2-receptor-selective (“CB2-receptor-selective agonist”).

Lower urinary tract dysfunction (“LUTD”) is characterized by symptoms, signs, urodynamic observations and conditions associated with the lower urinary tract and urodynamic studies. LUTD includes, but is not limited to: lower urinary tract symptoms (“LUTS”), which includes overactive bladder (“OAB”); signs such as measurements of aspects of LUTS; urodynamic observations such as detrusor overactivity (“DO”); and conditions such as acute retention of urine and benign prostatic obstruction. (Abrams, et al., “The Standardisation of Terminology in Lower Urinary Tract Function: Report from the Standardisation Sub-Committee of the International Continence Society,” Urology 61:37-49 (2003)).

An exemplary aspect of the invention involves the treatment of one or more symptoms, signs, urodynamic observations and conditions of LUTD with a CB2 receptor-selective-agonist.

LUTS is divided into three groups: storage, voiding and post micturition symptoms. Storage symptoms occur during the storage phase of the bladder and include increased daytime frequency, urinary incontinence, urgency and nocturia. Voiding symptoms are experienced during the voiding phase and include slow stream and hesitancy. Post micturition symptoms are experienced immediately after micturition, and include symptoms such as feeling of incomplete emptying and post micturition dribble. LUTS also includes symptoms associated with sexual intercourse and pelvic organ prolapse. LUTS further includes genital and lower urinary tract pain. Pain may be related to bladder filling or voiding, may be felt after micturition or may be continuous. Bladder pain is felt suprapubically or retropubically and usually increases with bladder filling. Bladder pain may persist after voiding. Genital and lower urinary tract pain further include urethral pain, vulval pain, vaginal pain, scrotal pain, perineal pain and pelvic pain. Painful bladder syndrome is the complaint of suprapubic pain related to bladder filling, accompanied by other symptoms such as increased daytime and night-time frequency, in the absence of proven urinary infection or other obvious pathology. (Abrams, et al. at 40.)

OAB refers to urgency, with or without urge incontinence, and often with frequency and nocturia. (Abrams, et al. at 40.) Such symptoms are suggestive of urodynamically demonstrable detrusor overactivity.

Signs of LUTD include measurements of daytime frequency, nocturia, 24-hour frequency, polyuria, nocturnal urine volume, nocturnal polyuria and maximum voided volume from frequency volume charts and bladder diaries. Signs further include physical examination, such as abdominal examination and perineal/genital inspection, urinary incontinence experienced during examination and stress urinary incontinence.

Urodynamic observations of LUTD include observations of the detrusor. Detrusor, or m. detrusor urinae, refers to the smooth muscle structure of the bladder. In normal detrusor function, bladder filling with little or no change in pressure is allowed. Also, in normal detrusor function, no involuntary phasic contractions occur, even with provocation. DO is a urodynamic observation that is characterized by involuntary detrusor contractions during the filling phases which may be spontaneous or provoked. (Abrams, et al. at 44.) One pattern of DO includes detrusor overactivity incontinence (“DO incontinence”). DO incontinence refers to incontinence resulting from an involuntary detrusor contraction.

Urodynamic observations of LUTD further include filling cystometry and pressure flow studies of voiding. Pressure flow studies can measure detrusor and urethral function during voiding. Abnormal urethral function can involve bladder outlet obstruction. Bladder outlet obstruction refers to obstruction during voiding. It is characterized by increased detrusor pressure and reduced urine flow rate.

Conditions of LUTD are the presence of urodynamic observations associated with characteristic symptoms or signs and/or non-urodynamic evidence of relevant pathological processes. (Abrams, et al. at 38.) A condition of LUTD, acute retention of urine, is painful, palpable or percussible bladder, when the patient is unable to pass any urine. A further condition of LUTD, benign prostatic obstruction is a form of bladder outlet obstruction.

An exemplary embodiment of the present invention involves a method for treating overactive bladder, lower urinary tract symptoms or detrusor overactivity in a patient by administering a CB2-receptor-selective agonist to a patient.

Cannabinoids and their derivatives exert their effects via cannabinoid receptor type 1 (“CB1”), mostly expressed in brain and responsible for cannabinoid psychoactivity, and cannabinoid receptor type 2 (“CB2”), expressed by peripheral sensory neurons and in the immune system. A cannabinoid can exhibit different degrees of selectivity for one or both receptors. CB2-receptor-selective agonists are compounds that exert their effects by selectively activating cannabinoid receptor type 2. As used herein, CB2-receptor-selective agonists include any natural, synthetic or derivative CB2-receptor-selective agonist compound, prodrugs of CB2-receptor-selective agonists and metabolites of CB2-receptor-selective agonists. CB2-receptor-selective agonists can also be peripherally selective, which involves a mode of action at the peripheral nervous system or in peripheral tissues rather than in the central nervous system. CB2-receptor-selective agonists as used herein include both peripherally selective and non-peripherally selective agonists. An aspect of the invention involves administration of a peripherally selective CB2-receptor-selective agonist.

CB2-receptor-selective agonists can include plant-derived or animal-derived cannabinoid or cannabimimetic compound selected from the group of aminoalkylindoles, anandamides, 3-aroylindoles, aryl and heteroaryl sulfonates, arylsulphonamides, benzamides, biphenyl-like cannabinoids, cannabinoids optionally further substituted by fused or bridged mono- or polycyclic rings, pyrazole-4-carboxamides, eicosanoids, dihydroisoindolones, dihydrooxazoles, α-pinene derivatives, quinazolinediones, quinolinecarboxylic acid amides, resorcinol derivatives, tetrazines, triazines, pyridazines and pyrimidine derivatives, and analogues and derivatives thereof. In one aspect of the instant invention, the CB2-receptor-selective agonist is an (+)α-pinene derivative. In a further aspect of the invention, the CB2-receptor-selective agonist is a benzofuran derivative.

One exemplary embodiment of the instant invention involves administration of a compound of Formula I:

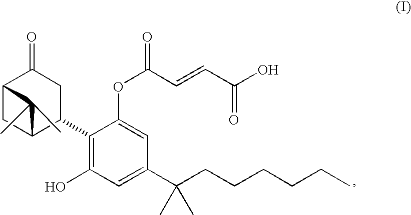

or a pharmaceutically acceptable salt, ester or solvate thereof.

The compound of Formula I and its synthesis are described in WO 2003/063758 (Compound Z) (see, e.g., p. 40, Scheme 13) and in U.S. Patent Publication No. 2005/0020544 (Compound Z) (see, e.g., p. 19, Scheme 13). The compound of Formula I is commercially known as Cannabinor (Pharmos Corporation) and is peripherally selective.

A further exemplary embodiment involves administration of a compound of Formula II:

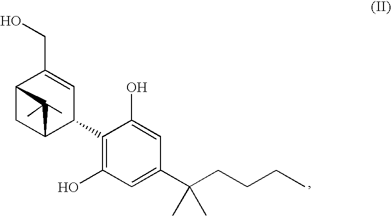

or a pharmaceutically acceptable salt, ester or solvate thereof.

The compound of Formula II is generally disclosed in WO 2003/063758. In particular, a precursor of the compound of Formula II and its synthesis are described in WO 2003/063758 (Compound AH) (see, e.g., p. 46, Scheme 21). It is understood that one of skill in the art would understand how to make a compound of Formula II from Compound AH.

Another embodiment of the instant invention involves administration of a compound of Formula III:

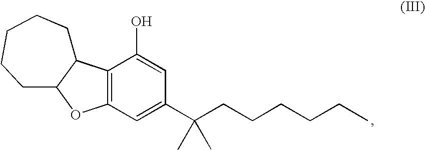

or a pharmaceutically acceptable salt, ester or solvate thereof.

The compound of Formula III and its synthesis are described in WO 2006/129318 (C7S-1) (see, e.g. Example 1; p. 42).

A further exemplary embodiment of the instant invention involves administration of a compound of Formula IV:

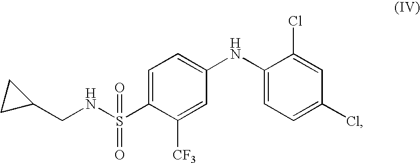

or a pharmaceutically acceptable salt, ester or solvate thereof.

The compound of Formula IV and its synthesis are described in U.S. Prov. Appl. No. 60/875,536 (Compound 3). The compound of Formula IV was prepared as follows:

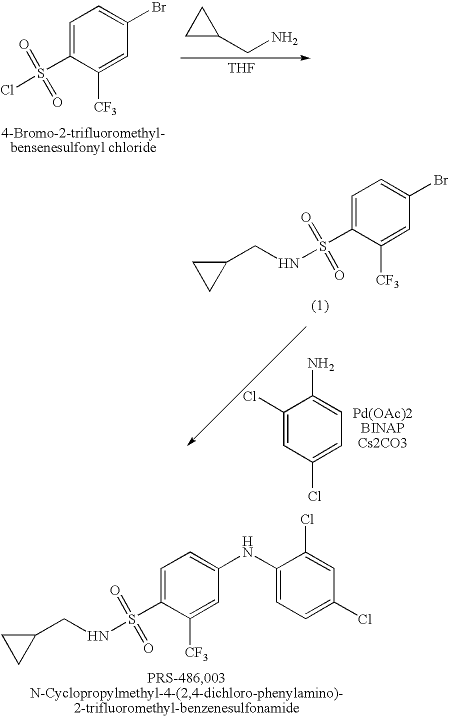

### Synthesis of the Compound of Formula IV: N-Cyclopropylmethyl-4-(2,4-dichloro-phenylamino)-2-trifluoromethyl-benzenesulfonamide

a) To a solution of cyclopropylamine (210 mg. 2.95 mmol) and in 15 ml THF 4-20 bromo-2-trifluoromethylbenzenesulfonyl chloride (321 mg, 0.99 mmol) was added in one portion and reaction mixture was stirred for a day. Ethyl acetate was added and mixture was washed twice with 1 N HCl, water and brine. After drying over sodium sulfate solvent was evaporated and crude solid used in the next stage. Yield 97%. b) Mixture of the product of step (a) (356 mg, 0.97 mmol), 2,4-dichloroaniline (270 mg, 1.66 mmol), Pd(OAc)₂ (23 mg, 0.10 mmol), BINAP (69 mg, 0.11 mmol) and cesium carbonate (334 mg, 1.04 mmol) in 15 ml toluene was reflaxed for 6 hours. Toluene was evaporated in vacuum and crude oil was purified by Combiflash (PE-THF). Yield 56%.

¹H NMR: (CDCl₃) δ: 0.09-0.13 (2H, m, CH); 0.45-0.51 (2H, m, CH); 0.89 (1H, m, CH); 2.81-2.85 (2H, dd, CH); 4.68 (1H, s, NH); 6.19 (1H, s, NH); 7.16-7.19 (1H, dd, CH); 7.24-7.28 (3H, m, CH); 7.32-7.39 (2H, m, CH); 7.51 (1H, d, CH); 8.09 (1H, d, CH). Molecular ion observed [M-H]+=439 consistent with the molecular formula C17H₁₅Cl₂F₃N₂O₂S.

A further embodiment of the invention comprises administering a combination of two or more compounds of Formula I, II, III or IV for the treatment or prevention of lower urinary tract dysfunction.

The compounds of the invention may contain one or more asymmetric carbon atoms and thus may occur as racemates and racemic mixtures, a single enantiomer, mixtures of enantiomers, diastereomeric mixtures and individual diastereoisomers. All such isomeric forms of these compounds are included in the present invention. Each stereogenic carbon may be of the R or S configuration. These mixtures of enantiomers and diastereomers can be separated into stereoisomercially uniform components in a known manner or synthesized a priori as separate enantiomers.

Further aspects of the invention include administration of a pharmaceutically acceptable salt of a compound of the invention. Pharmaceutically acceptable salts include those derived from pharmaceutically acceptable inorganic or organic acids and inorganic or organic bases, including amino acids. For example, such salts may be formed by any carboxy or sulfo groups present in the molecule.

Pharmaceutically acceptable acid addition salts of the compounds include salts derived from inorganic acids such as hydrochloric, nitric, phosphoric, sulfuric, hydrobromic, hydriodic, phosphorous, and the like, as well as salts derived from organic acids such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include, but are not limited to, sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are salts of amino acids such as arginate and the like and gluconate or galacturonate (Berge S. M. et al., J. of Pharmaceutical Science, 66: 1-19 (1977)).

The acid addition salts of said basic compounds are prepared by contacting the free base form with a sufficient amount of the desired acid to produce the salt in the conventional manner. The free base form may be regenerated by contacting the salt form with a base and isolating the free base in the conventional manner. The free base forms may differ from their respective salt forms in certain physical properties such as solubility in polar solvents.

Pharmaceutically acceptable acid addition salts of the compounds include salts derived from bases, and thus such salts include, but are not limited to, sodium, potassium, calcium, meglumine, magnesium, diethylamine, silver, procaine, piperazine, lysine, diethanolamine, cholinate, benzathine and tromethamine.

The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms may differ from their respective salt forms in certain physical properties such as solubility in polar solvents.

Esters of the compounds encompassed by the instant invention include compounds that contain esters including, but not limited to, alkyl, cycloalkyl, heteroalkyl, heterocycloalkyl, alkenyl, alkynyl, cycloalkenyl, heterocycloalkenyl, heteroalkenyl and heteroalkynyl esters.

Solvates of the compounds encompassed by the instant invention include compounds of the invention that have a physical association with one or more solvent molecules. This physical association involves varying degrees of ionic bonding, including hydrogen bonding. In some instances, the solvate is capable of isolation. Solvates encompass both solution-phase and isolatable solvates. Solvates include, but are not limited to, ethanolates and methanolates. A hydrate is a solvate wherein the solvent molecule is water. The instant invention includes hydrates of compounds described herein.

It should be understood that the present invention encompasses, by example and without limitation, the CB2-receptor-selective agonists disclosed herein; acid-addition salts of the CB2-receptor-selective agonists; basic addition salts of the CB2-receptor-selective agonists; solvates of the CB2-receptor-selective agonists; and esters of the CB2-receptor-selective agonists. Accordingly, it should be understood that the present invention includes, by example and without limitation, compounds of any one of Formulas I, II, III or IV; acid-addition salts of such compounds or base addition salts of such compounds; solvates of such compounds; and esters of such compounds.

Prodrugs are compounds which are rapidly transformed in vivo to a parent compound of the instant invention, for example, by hydrolysis in the blood. An aspect of the instant invention therefore encompasses prodrugs of CB2-receptor-selective agonists. A still further aspect of the invention includes prodrugs of a compound of one of Formulas I, II, III or IV. Prodrugs can be useful in instances in which they are easier to administer than the parent drug. They may, for instance, be bioavailable by oral administration in instances in which the parent drug is not. The prodrug may also have improved solubility compared to the parent drug in pharmaceutical compositions. All of these pharmaceutical forms are intended to be included within the scope of the present invention.

As used herein, the phrase “therapeutically effective amount” refers to that amount of CB2 receptor-selective agonist that provides a therapeutic benefit in the treatment or management of LUTD, including prevention of LUTD.

The magnitude of a therapeutic, including prophylactic, dose of a CB2-selective agonist in the management of LUTD will vary with the severity of the syndrome and the route of administration. The dose, and perhaps the dose frequency, will also vary according to the age, body weight, gender, medical condition, concurrent treatment, if any, and response of the individual patient.

Effective doses may be extrapolated from dose-response curves derived from in vitro or animal model test systems in combination with pharmacokinetic data.

Further, the dose of the compounds of the instant invention for humans can be determined by standard clinical techniques. In one embodiment of the present invention, the dose is generally in the range of from 0.01 mg to about 50 mg per kg body weight, in a regimen of 1-4 times a day.

An exemplary embodiment of the invention is directed to a method of administering a compound of the invention in a range of from about 0.1 mg to about 20 mg per kg body weight.

In a further exemplary embodiment, the compound of Formula I is administered in a total daily dose range for the conditions described herein, in an amount of about 10 mg to about 300 mg daily dose for oral administration; about 3 mg to about 24 mg for intravenous administration; and about 10 mg to about 100 mg for subcutaneous administration. In a still further aspect of the invention, the compound of Formula I is administered in a total daily dose range for overactive bladder, in an amount of about 10 mg to about 300 mg daily dose for oral administration; about 3 mg to about 24 mg for intravenous administration; and about 10 mg to about 100 mg for subcutaneous administration.

In a further exemplary embodiment of the invention, the compound of Formula III is administered in a total daily dose range for the conditions described herein, in an amount of about 10 mg to about 300 mg daily dose for oral administration; about 3 mg to about 24 mg for intravenous administration; and about 10 mg to about 100 mg for subcutaneous administration. In a still further aspect of the invention, the compound of Formula III is administered in a total daily dose range for overactive bladder, in an amount of about 10 mg to about 300 mg daily dose for oral administration; about 3 mg to about 24 mg for intravenous administration; and about 10 mg to about 100 mg for subcutaneous administration.

Any suitable route of administration may be employed for providing the patient with an effective dosage of a CB2-receptor-selective agonist according to the methods of the instant invention. For example, oral, intravenous (“IV”), transdermal, nasal, rectal, parenteral, subcutaneous, intramuscular, aerosol, topical, ocular, inhalation, intraperitoneal, intrathecal, intravesicle, rectal, vaginal and like forms of administration may be employed. Dosage forms include patches, tablets, nasal sprays, injections, IVs, troches, dispersions, suspensions, solutions, capsules and further like dosage forms.

In one embodiment of the invention, a dose of a CB2-receptor-selective agonist is provided in a tablet by oral administration. In a further embodiment of the invention, the compound of Formulas I, II, III or IV is provided in a tablet by oral administration. In a still further embodiment of the invention, the compound of Formula III is provided in a tablet by oral administration.

In a further exemplary embodiment of the invention, the compound of Formula III is administered for LUTD in an amount of about 10 mg to about 300 mg daily dose in a tablet for oral administration. In a still further exemplary embodiment of the invention, the compound of Formula III is administered for overactive bladder in an amount of about 10 mg to about 300 mg daily dose in a tablet for oral administration.

It should be understood that the phrase “therapeutically effective amount of a CB2-receptor-selective agonist” is encompassed by the above-described dosage amounts and dose frequency schedule. It should be further understood that doses for CB2-receptor-selective agonists other than the compound of Formula I that are therapeutically equivalent to the above-described dosages for the compound of Formula I fall within the scope of the instant invention.

A further embodiment of the invention involves pharmaceutical compositions comprising a CB2-receptor-selective agonist, such as a compound of one of Formulas I, II, III or IV, an active ingredient, and that also may contain a pharmaceutically acceptable carrier, and optionally, other therapeutic ingredients. A pharmaceutically acceptable carrier involves a non-toxic carrier or adjuvant that may be administered to a patient, together with one or more compounds of the present invention, and which does not destroy the pharmacological activity thereof.

Solid pharmaceutical compositions of the instant invention for oral administration as tablets, pills, capsules, softgels or the like may be prepared by mixing a compound of the invention with conventional, pharmaceutically acceptable ingredients such as corn starch, lactose, sucrose, mannitol, sorbitol, talc, polyvinylpyrrolidone, polyethyleneglycol, cyclodextrins, dextrans, glycerol, polyglycolized glycerides, tocopheryl polyethyleneglycol succinate, sodium lauryl sulfate, polyethoxylated castor oils, non-ionic surfactants, stearic acid, magnesium stearate, dicalcium phosphate and gums as pharmaceutically acceptable diluents. The tablets or pills can be coated or otherwise compounded with pharmaceutically acceptable materials known in the art, such as microcrystalline cellulose and cellulose derivatives such as hydroxypropylmethylcellulose (HPMC), to provide a dosage form affording prolonged action or sustained release. Coating formulations can be chosen to provide controlled or sustained release of the drug, as is known in the art.

Other solid pharmaceutical compositions of the present invention can be prepared such as suppositories or retention enemas, for rectal administrations using conventional suppository bases such as cocoa butter or other glycerides.

Liquid pharmaceutical compositions of the instant invention may be prepared for oral administration. The liquid compositions include aqueous solutions, with or without organic cosolvents, aqueous or oil suspensions including, but not limited, to cyclodextrins as suspending agent, flavored emulsions with edible oils, triglycerides and phospholipids, as well as elixirs and similar pharmaceutical vehicles. In addition, the pharmaceutical compositions of the present invention may be formed as aerosols, for buccal and oropharyngeal administration. The aerosol is conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator can be formulated containing a powder mix of the compound and a suitable powder base such as lactose or starch.

Topical pharmaceutical compositions of the present invention can be formulated as solutions, lotion, gel, cream, ointment, emulsion or adhesive film with pharmaceutically acceptable excipients including but not limited to propylene glycol, phospholipids, monoglycerides, diglycerides, triglycerides, polysorbates, surfactants, hydrogels, petrolatum or other such excipients as are known in the art.

Pharmaceutical compositions of the present invention may be manufactured by processes well known in the art, e.g., by means of conventional mixing, dissolving, granulating, grinding, pulverizing, dragee-making, levigating, emulsifying, encapsulating, entrapping or lyophilizing processes.

Pharmaceutical compositions of the present invention may also include one or more additional active ingredients. In particular, the CB2-receptor-selective agonists of the invention may be coadministered or used in combination with one or more other drugs used in the treatment of overactive bladder. The administration and dosage of such additional active ingredients is according to the schedule listed in the product information sheet of the approved active ingredient, in the Physicians Desk Reference (“PDR”) as well as therapeutic protocols well known in the art.

When two or more active ingredients are administered to achieve the therapeutic goals of the present invention, co-administration can be in a unique dosage form for the combination or in separate dosage forms for combined administration. Combined administration in the context of this invention is defined to mean the administration of more than one therapeutic in the course of a coordinated treatment to achieve an improved clinical outcome. Such combined administration may occur at the same time and also be coextensive, that is, occurring during overlapping periods of time, or occur at different times.

An exemplary embodiment of the instant invention encompasses a combined administration of a CB2-receptor-selective agonist with a muscarinic antagonist. A further exemplary embodiment of the invention is a dosage form comprising darifenacin and a CB2-receptor-selective agonist.

The invention is further defined by reference to the following examples that further describe the method of the present invention, as well as its utility. It will be apparent to those skilled in the art that modifications, both to materials and methods, may be practiced which are within the scope of the invention.

### EXAMPLES

### Example 1

### Physicochemical Properties

Information regarding certain physicochemical properties of the compounds of Formulas I, II, III and IV are presented in the following Table. Expected water solubility, logP and logD at pH 7 were calculated using Advanced Chemistry Development software (ACD labs, version 4.04). THC and Nabilone, being both commercially available cannabinoids, are reported herein as references.

TABLE 1
Selected physicochemical properties

| Compound | Structure | MW | Solubility | | LogP | LogD, pH7 |
|---|---|---|---|---|---|---|
| Δ⁹-THC | 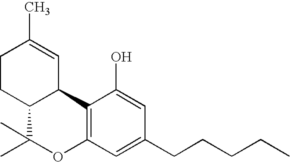 | 314.46 | 400 | ng/ml | 7.68 ± 0.35 | 7.68 |
| Nabilone | 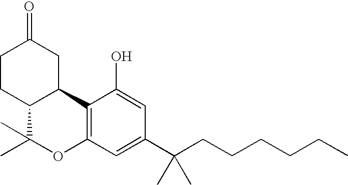 | 372.54 | 8 | ng/ml | 7.01 ± 0.39 | 7.01 |
| (I) | (described herein) | 470.60 | >10 | mg/ml | | 2.5 |
| (II) | (described herein) | 358.51 | | | 6.5 | |
| (III) | (described herein) | 330.26 | | | 7.9 | |
| (IV) | (described herein) | 439.28 | | | 6.8 | |

The actual solubility of certain compounds of the invention was assessed in aqueous buffers and final concentration of compounds was determined using HPLC and spectrophotometer methodologies.

### Example 2

### Binding Affinity for the CB1 and CB2 Receptors

The CB1 and CB2 binding assays were performed as described in International Patent Applications WO 06/129318 and results, expressed as IC₅₀ in nM, are reported in the following table. Binding affinity is represented by an IC₅₀ value, which is the concentration of a test compound that will displace 50% of a radiolabeled agonist from the CB receptors.

TABLE 2
IC₅₀ (nM) of compounds of formula (I), (II) and (III)
Evaluation of therapeutic effects of the compounds of
Formulas I, II, III and IV was carried out in experiments to show
the use of these compounds as agents for the treatment of
LUTD. These effects were evaluated as set forth below.

| | | | CB₂/CB₁ |
|---|---|---|---|
| | | | affinity |
| Compound | CB₂ IC₅₀ | CB₁ IC₅₀ | ratio |
| THC* | 36.4 | 40.7 | 0.89 |
| (I) | 19.4 | 660 | 34 |
| (II) | 8.4 | 181 | 21 |
| (III) | 6.5 | 92 | 14 |
| (IV) | 30 | 313 | 10 |

### Example 3

### Effect of Compounds on Cystometry in an Acetic Acid Model

This study involved an assessment of the acute effect of the compounds of Formulas II, III and IV, which exhibit varying degrees of CB2/CB1 affinity (Table 2), on bladders in urethane-anesthetized rats in an acetic acid model using cystometry. As used herein, the term “acute” means that the animals were dosed only one time, 30 minutes prior to taking the cystometry measurements (i.e., 30 minutes prior to the observation period). The acetic acid-induced model is an irritative model in which increased micturition frequency (decreased micturition interval) and reduced bladder capacity cystometry may be obtained after an intravesical infusion of a dilute solution of acetic acid (McMurray G, Casey J H, Naylor A M. Animal models in urological disease and sexual dysfunction. Br J Pharmacol. 2006; 147 Suppl:S62; Kakizaki H, de Groat W C. Role of spinal nitric oxide in the facilitation of the micturition reflex by bladder irritation. J Urol 1996; 155:355).

However, one of the potential complications in the interpretations of the relevance in predicting success in the clinic is that the model also involves an inflammatory component (McMurray G, Casey J H, Naylor A M. Animal models in urological disease and sexual dysfunction. Br J Pharmacol. 2006; 147 Suppl:S62), and drugs with known anti-inflammatory mechanisms may also exhibit efficacy that could be mistaken for an effect on the sensory arm of the micturition reflex. This might be relevant in the comparison of compounds with varying selectivity for the CB2 vs. CB1 receptors, as CB2 receptor agonists are expected to possibly possess an anti-inflammatory effect (Klein T W. Cannabinoid-Based Drugs as Anti-Inflammatory Therapeutics. Nat Rev Immunol. 2005; 5:400), consistent with the localization of the CB2 receptor on cells of the immune system. Another potential complication of this model is the use of urethane anesthesia, and the known interactions of cannabinoid agonists possessing CB1 activity with the vasculature in animals subjected to urethane anesthesia (Kwolek G, Zakrzeska A, Schlicker E, Gothert M, Godlewski G, Malinowska B. Central and peripheral components of the pressor effect of anandamide in urethane-anaesthetized rats. Br J Pharmacol. 2005; 145:567). When animals are treated with CB1 agonists under urethane anesthesia, the vasodilation effects of the CB1 agonists are exacerbated, and may lead to production of compensatory reflex mechanisms that could ultimately influence some of the urodynamic parameters that are measured. The above should be considered when interpreting data from this particular model.

Animals

Adult female Wistar rats (Janvier, Le Genest Saint Lisles, France), weighing 225-250 g were used. They were delivered to the laboratory at least 5 days before the experiments in order to be acclimatized to laboratory conditions, and were given food (Teklad Global 16% Protein Rodent Diet, Harlan, Gannat, France) and water ad libitum. The animal room was maintained at 21-24° C. with a 12/12 hours alternating light-dark cycle (light phase 07:00-19:00).

Bladder and Intravenous (IV) Catheter Implantation

Rats were anesthetized by intraperitoneal injection of urethane (1.5 g/kg). After making a midline incision of the abdomen, a polyethylene catheter (0.3 mm ID and 0.7 mm OD) was implanted in the bladder through the dome. An IV catheter (0.58 mm ID and 0.96 mm OD) was also introduced into a jugular vein for test substance administration.

CB2-Receptor-Selective Agonists Preparation and Administration

CB2-receptor-selective agonists (compounds of Formulas II, III and IV) were initially dissolved in a vehicle containing a co-solvent mixture of Cremophor EL® (70%, BASF Corporation, Florham Park, N.J.) and ethanol (30%) as a 20-fold concentrated stock solution of the highest dose for testing. For lower doses, individual 20× stock solutions in Cremophor EL® (70%) and ethanol (30%) were also prepared by further dilution of the original 20× stock solution. Just prior to use, the appropriate 20× compound stock solution or vehicle was diluted 1:20 dilution (v:v) with saline to give the appropriate final concentration for dosing, and a single test substance or its vehicle were administered intravenously in a volume of 5 ml/kg and at an infusion rate of 0.2 ml/min.

Cystometry

Cystometric investigations were performed in the urethane anesthetized rats after bladder and jugular vein catheter implantation surgery. The bladder catheter was connected via a T-tube to a MX 860 Novatrans III Gold strain gauge (Medex Medical SARL, Nantes-Carquefou, France) and to a single-syringe infusion pump (70-2208 Model II Plus, Harvard Apparatus; Les Ulis, France). Physiological saline or 0.2% acetic acid (AA) in saline was infused into the bladder at a constant flow rate (0.05 ml/min), and intravesical pressure was recorded continuously using a MacLab/8e interface (ADInstruments Pty Ltd., Castle Hill, Australia) and the Chart software program. After a control period of 30 minutes (to calculate basal values), a single test substance or its vehicle were administered IV and the intravesical pressure was recorded for 1 hour after compound administration.

Cystometry Measurements and Analysis

Intravesical pressure data were analyzed with Microsoft Excel software. The micturition parameters measured were the following: Basal Pressure (“BP,” mmHg); Threshold Pressure (“ThP,” pressure at which micturition occurs, mmHg); Amplitude of Micturition (“AM,” pressure between ThP and Micturition Pressure, mmHg); Intercontraction Interval (“ICI,” time between two subsequent micturitions, sec); Micturition Frequency (“MF,” number of micturition contractions per 15 minute interval); and Area Under the Curve (“AUC” per 15 minute interval, mmHg·sec) was calculated from the lowest intravesical pressure value during the 15 minute interval. Bladder capacity (“BC”) was measured in ml.

For each rat, basal values for each parameter were calculated as the mean of the last four micturitions before vehicle or test substance administration. Results were expressed as % variation between basal values and values obtained per 15 minute interval (AUC, MF) or values measured at 20, 40 and 60 minutes after vehicle or test substance administration (AM, BP, ThP, BC, ICI). The results were given as mean values standard error of the mean (SEM). Statistical analyses were performed using GraphPadPrism® 4.02. An unpaired Student t-test was used to compare basal values of the 0.9% NaCl and 0.2% AA groups. A one-way ANOVA with repeated measures followed by Newman-Keul's test was used for comparison between basal values and the treatment period in each group. A One-way ANOVA followed by Newman-Keul's test was used for comparison of % variation of each cystometric parameter and each time between different groups. A p<0.05 was accepted for statistical significance.

Rats were excluded from the study in the following cases: (1) the mean value of the four ICI evaluated before injection of the vehicle or test substance was greater than 150 sec for the 0.2% AA infusion group; (2) the basal bladder pressure was higher than 15 mmHg; (3) an absence of a micturition reflex; and (4) the variation of the four individual values for the ICI before vehicle or test substance administration was greater than 25%.

Effect of Compounds of Formulas II, III and IV on Micturition in Urethane-Anesthetized Rats in the Acetic Acid Bladder Hyperactivity Model

Infusion of 0.2% acetic acid (“AA”) into the bladder induced a marked 40% decrease in inter-contraction interval (ICI) in comparison to saline-infused rats (124.7±10.7 sec for 0.2% AA and 207.6±27.3 sec for saline), and a similar reduction (40%) was also seen for BC. Conversely, MF was significantly increased in the 0.2% AA-infused rats compared to saline-infused rats (6.0±0.6 peaks/15 min for 0.2% AA and 4.1±0.4 peaks/15 min for saline. No significant differences between the 0.2% AA and saline-infused bladder groups were observed for any other cystometric parameters measured, and the only significant effect of the intravenous administration of the vehicle in comparison to basal values was a 16% and 15% of reduction in the amplitude of micturition (AM) at 40 and 60 min after vehicle administration (p<0.05).

Effect of the Compound of Formula II on Micturition

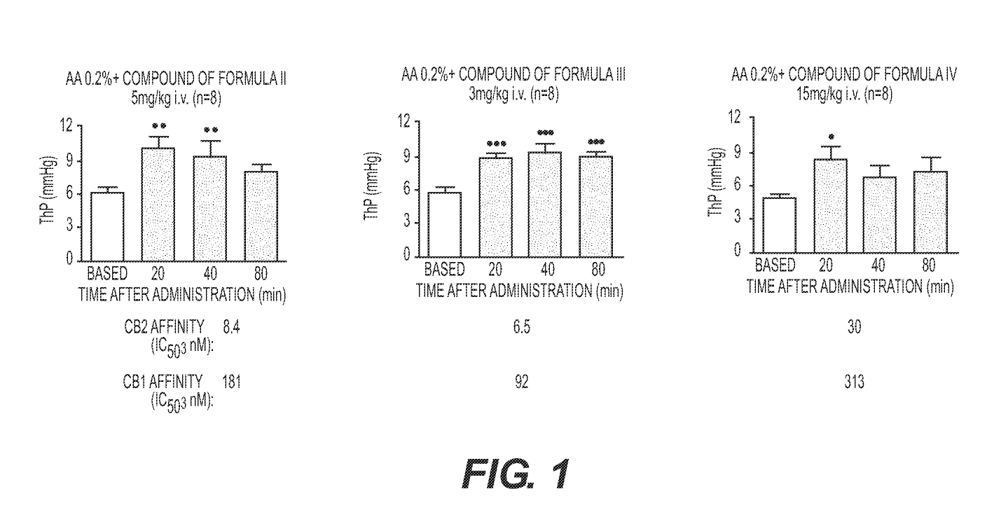

The compound of Formula II had no effect on ICI at any tested dose (0.5, 1.5, and 5 mg/kg, i.v.) or time (20, 40, or 60 min) after administration compared to basal values. Significant increases in ThP were detected at 20, 40 and 60 min after 0.5 mg/kg, 40 min after 1.5 mg/kg, and at 20 and 40 min after 5 mg/kg compound of Formula II (p<0.05). The effect on ThP for the 5 mg/kg dose is shown in FIG. 1. After the 5 mg/kg dose, basal pressure (BP at 20 min after administration), was also significantly increased (p<0.05).

Effect of the Compound of Formula III on Micturition

The compound of Formula III had no effect on ICI at any tested dose (0.3, 1, and 3 mg/kg, i.v.) or time (20, 40, or 60 min) after administration compared to basal values. ThP was significantly increased compared to basal values after the 3 mg/kg dose at all time points, and the effect on ThP for this dose is shown in FIG. 1. BP was also significantly increased (from 0 to 60 min) after administration of 3 mg/kg (p<0.05).

Effect of the Compound of Formula IV on Micturition

The compound of Formula IV had no effect on ICI at any tested dose (1.5, 5, and 15 mg/kg, i.v.) or time (20, 40, or 60 min) after administration compared to basal values. A significant increase in ThP was observed at 20 min after administration of 15 mg/kg in comparison to basal values, and the effect on ThP for this dose is shown in FIG. 1. BP was not changed after any dose or time point compared to basal values.

The results above show that the effect on ThP is likely mediated by the CB2 receptor. FIG. 1 sets forth CB2 affinity and CB1 affinity for each of the compounds of Formulas II, III and IV. The animals were dosed according to CB2 affinity, so the CB2 receptor occupancy at each of the administered doses of the compounds were similar. However, the CB1 affinities for the compounds of Formulas II, III and IV were IC₅₀ of 181, 92 and 313 nM, respectively, and at the administered dose, varying degrees of CB1 receptor occupancy could be expected. Despite the varying CB1 occupancy at the administered dose, each of the compounds reached the same level of ThP. Thus, this demonstrates that the effect on ThP is likely mediated by the CB2 receptor.

### Example 4

### Cystometry in Normal Conscious Rats

This study involved a determination of the acute effect of the compound of Formula I, a peripherally-selective, CB2-receptor-selective agonist, on normal bladder function in normal conscious freely moving rats using cystometry.

Animals

Female Sprague-Dawley rats, weighing 225-300 g, were maintained under standard laboratory conditions with a 12:12 light/dark cycle and free access to food pellets and water. After surgical procedures, rats were individually caged to prevent chewing of the catheters. For surgical procedures, the rats were anesthetized by intraperitoneal injection of 75 mg/kg ketamine (Ketalar®, Pfizer, Sweden) and 15 mg/kg xylazine (Rompun®, Bayer, Sweden).

Bladder Catheter Implantation

The bladder was exposed via a mid-line incision of the lower abdomen. A saline-filled polyethylene catheter (PE-50, Clay-Adams, Parsippany, N.J.) with a cuff was inserted into the dome of the bladder and held in place with a purse string suture. The catheter was tunneled subcutaneously and anchored at the neck with a silk ligature, and the free end of the catheter was sealed.

Intravenous (IV) Catheter Implantation

In the same surgical session as for implantation of the bladder catheter, access to the femoral vein was given via the mid-line incision of the lower abdomen after careful blunt dissection of the subcutaneous fascias and the femoral vascular sheath. A stretched PE-50 catheter filled with heparinized saline was introduced into the femoral vein and forwarded with its tip in the iliac vein. The catheter was then secured in position by two ligatures, tunneled subcutaneously to the skin of the back and anchored as described above, and the free end of the catheter was sealed.

Compound of Formula I Preparation and Administration

The appropriate solution of the compound of Formula I was prepared on the day of the experiment using phosphate-buffered saline (“PBS”) as the vehicle. The compound of Formula I was dissolved to obtain a 10 mg/ml stock solution. For the 3 mg/kg dose, each animal was administered individually weight-controlled amounts in a volume of approximately 100 μl IV of the stock solution. For lower doses, appropriate dilutions of the stock solution were made and animals were administered similar volumes containing 0.3 or 1 mg/kg. PBS was given IV at the same volume as the test compound to ensure that administration alone did not cause effects on micturition. After administration of the compound of Formula I, 100 μl of heparinized saline was also infused to flush the IV catheter, and a 90 to 120-minute observation period was allowed until final stable micturitions were obtained. Each animal served as its own control.

Conscious Cystometry

Cystometry was performed without anesthesia three days after the implantation of bladder catheters in normal rats. The conscious rats were placed in metabolic cages without restraint and the bladder catheters were connected via a T-tube to a pressure transducer (P23 DC; Statham Instruments Inc., Oxnard, Calif.) and a microinjection pump (CMA 100; Carnegie Medicine AB, Solna, Sweden). Micturition volumes were recorded with a fluid collector connected to a force displacement transducer (FT 03 D, Grass Instrument Co., Quincy, Mass.). Room-temperature saline was infused into the bladder continuously at a rate of 10 ml/h. Pressures and micturition volumes were recorded continuously with Acq Knowledge 3.8.1 software and a MP100 data acquisition system (Biopac Syst. Inc. Santa Barbara, Calif.) connected to a Grass polygraph (Model 7E, Grass Instrument Co). At the beginning of cystometry, the bladder was emptied via the bladder catheter.

Cystometry Measurements and Data Analysis

The following urodynamic parameters were investigated: micturition pressure (“MP;” maximum bladder pressure during micturition), threshold pressure (“ThP”; bladder pressure at start of detrusor contraction), flow pressure (“FP”; bladder pressure when urethra opens), micturition interval (“MI”; time between two micturitions), micturition volume (“MV”; volume of the expelled urine), and residual volume (“RV”; volume left in bladder after a micturition). At least a 30-minute period with stable and reproducible micturitions was recorded before drug administration and used as baseline value, to be compared with a corresponding stable 30-minute period after drug administration. For multiple comparisons, a one-way analysis of variance for repeated measures (Holm-Sidak) was used. Pairwise and non-pairwise comparisons were made by Student's t-test. All statistical calculations are based on the number of individual animals, and significant differences are accepted when p<0.05.
Rats were excluded from the study in the following case: 4 rats were not used due to postsurgical clotting of the intravenous femoral catheter.

Effect of the Compound of Formula I on Micturition in Conscious Normal Rats.

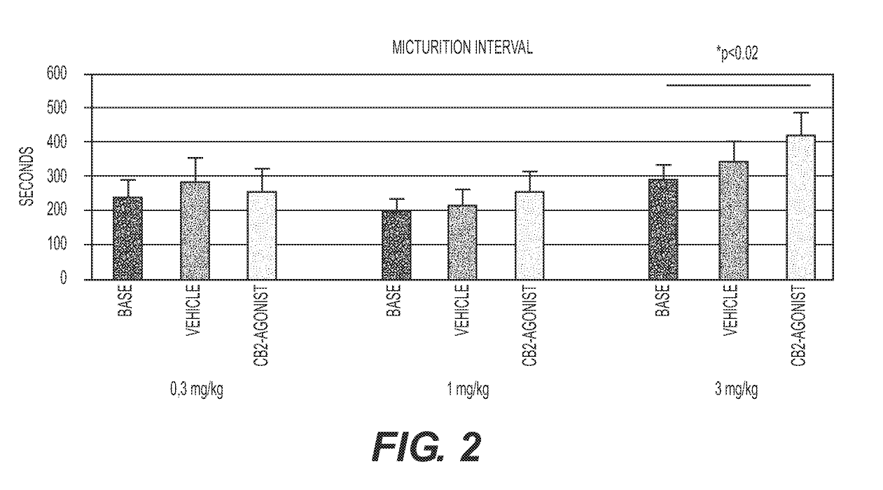

At baseline during continuous cystometries, MI was 240±55 sec for 0.3 mg/kg, 203±35 sec for 1 mg/kg, and 294±41 sec for the 3 mg/kg compound of Formula I groups, respectively (FIG. 2). After IV administration of the compound of Formula I at 0.3 mg/kg, 5 out of 8 rats responded with an increased MI. After 1 mg/kg compound of Formula I, 3 out of 8 rats showed an increased MI, and 7 out of 8 rats showed increased MI after 3 mg/kg compound of Formula I. At 0.3 mg/kg and 1 mg/kg compound of Formula I, MI was not significantly changed (260±69 sec and 251±63 sec, respectively). After 3 mg/kg compound of Formula I, a significant increase of MI to 423±63 sec (p<0.02 vs. baseline) was observed. Intravenous vehicle did not affect MI (284±76, 218±46, and 346±57 sec).

Basal pressure (“BP”) was 4.5±1.5 cmH₂O (for 0.3 mg/kg compound of Formula I), 3.4±1.3 cmH₂O (1 mg/kg), and 3.7±1.4 cmH₂O (3 mg/kg) at baseline. At all doses of the compound of Formula I, there was an even distribution of animals that responded with no change in BP (12.5-25% of rats), an increase in BP (25-37.5% of rats), or a decrease in BP (37.5-50% of rats). Overall, the compound of Formula I at 0.3 mg/kg, 1 mg/kg, or 3 mg/kg did not affect BP (4.7±2.0 cmH₂O, 3.7±1.2 cmH₂O, and 9.0±4.3 cmH₂O, respectively). After IV administration of vehicle, BP was not changed (3.9±1.6 cmH₂O, 2.8±1.3 cmH₂O, and 3.5±1.3 cmH₂O).

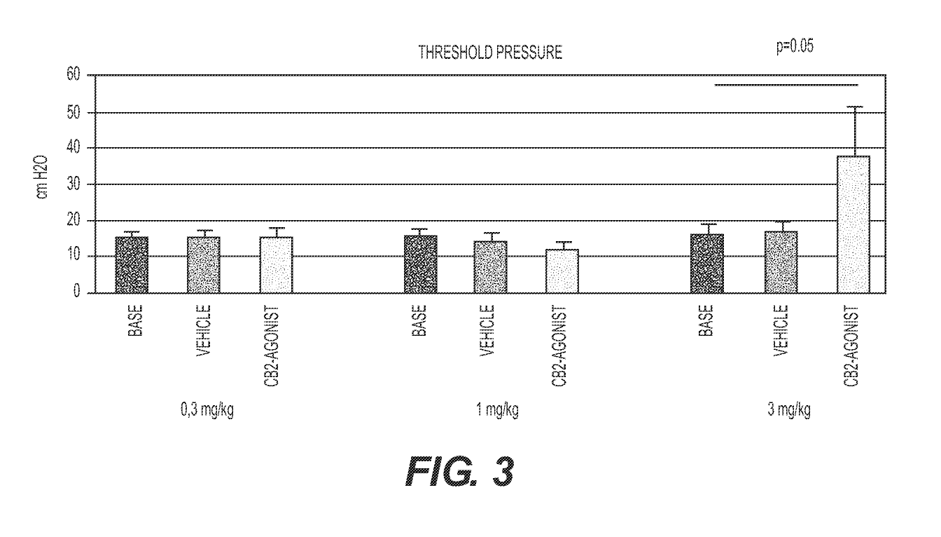

ThP was 15.4±1.7 cmH₂O (for 0.3 mg/kg compound of Formula I), 15.8±2.2 cmH₂O (1 mg/kg), and 16.5±2.5 cmH₂O (3 mg/kg) in basal recordings (FIG. 3). After administration of the compound of Formula I at 0.3 mg/kg, 5 out of 8 rats responded with increased ThP. An increase in ThP was seen in 1 out of 8 rats after 1 mg/kg and in 7 out of 8 rats after 3 mg/kg compound of Formula I. ThP was not significantly changed after administration of the compound of Formula I at 0.3 or 1 mg/kg, which yielded mean ThPs of 16.5±2.2 cmH₂O and 12.6±2.0 cmH₂O. After 3 mg/kg compound of Formula I, rats exhibited a trend towards higher ThP, which measured 38.1±13.9 cmH₂O (ANOVA ns; Paired t-test vs. baseline p=0.05; FIG. 3). After vehicle, ThP was unaltered (15.7±1.4 cmH₂O, 14.7±2.3 cmH₂O, and 17.5±2.5 cmH₂O).

Baseline FP measured 35.4±5.6 cmH₂O (0.3 mg/kg compound of Formula I), 27.6±3.5 cmH₂O (1 mg/kg), and 32.2±5.8 cmH₂O (3.0 mg/kg). At 0.3 and 1 mg/kg compound of Formula I, 50% of the animals responded with an increase or decrease in FP, without significant changes (34.1±8.8 cmH₂O and 26.0±2.4 cmH₂O, respectively). After IV administration of the compound of Formula I at 3 mg/kg, 6 out of 8 rats responded with an increased FP (62.3±18.4 cmH₂O), but the change was not significant. Intravenous vehicle did not affect FP (33.9±5.0 cmH₂O, 28.1±3.2 cmH₂O, and 32.2±5.4 cmH₂O).

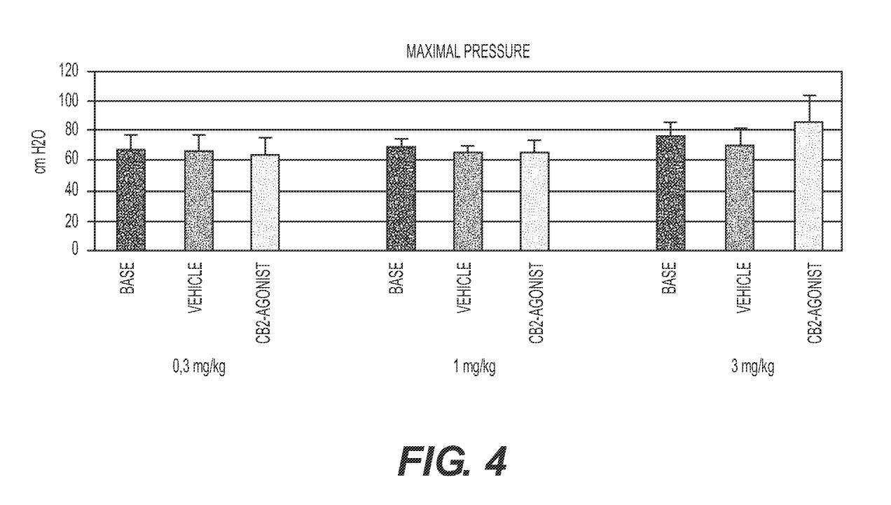

MP during baseline was 67.0±10.2 cmH₂O (0.3 mg/kg compound of Formula I), 68.7±7.1 cmH₂O (1 mg/kg), and 76.4±9.5 cmH₂O (3 mg/kg, FIG. 4). At all doses, the compound of Formula I did not affect MP (64.3±12.3 cmH₂O, 65.1±8.6 cmH₂O, and 87.3±18.9 cmH₂O, respectively at 0.3 mg/kg, 1 mg/kg, and 3 mg/kg). Vehicle administration did not affect MP (65.1±11.9 cmH₂O, 64.7±5.9 cmH₂O, and 70.6±11.1 cmH₂O).

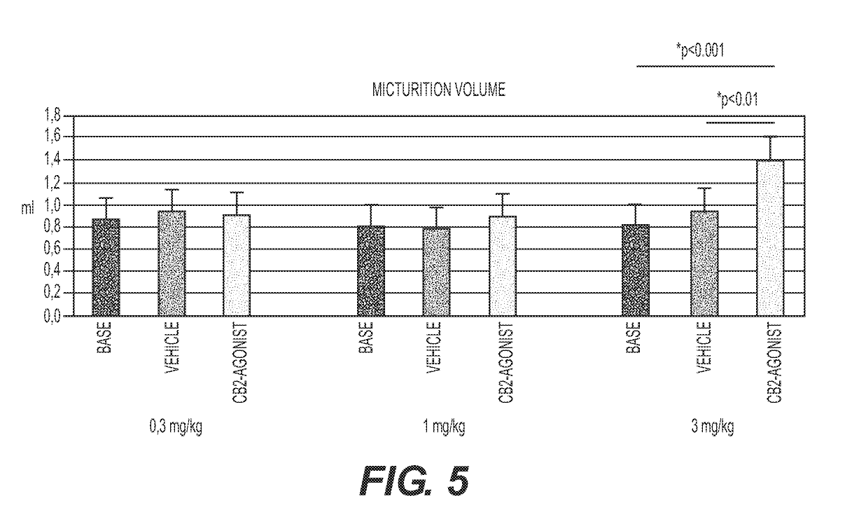

MV (FIG. 5) during baseline was 0.87±0.16 ml (0.3 mg/kg compound of Formula I), 0.82±0.17 ml (1 mg/kg), and 0.82±0.14 ml (3 mg/kg). After administration of the compound of Formula I at 0.3 mg/kg, 5 out of 8 rats responded with increased MV. After 1 mg/kg compound of Formula I, MV increased in 6 out of 8 rats and 7 out of 8 rats after 3 mg/kg. The mean MV levels were not significantly greater after administration of 0.3 mg/kg or 1 mg/kg compound of Formula I (0.93±0.21 ml and 0.90±0.17 ml, respectively), but after 3 mg/kg, a significant increase in MV was obtained (1.4±0.2 ml; p<0.001 vs. baseline, p<0.01 vs. vehicle). Vehicle did not affect MV (0.95±0.17 ml, 0.78±0.15 ml, and 0.96±0.16 ml).

There was no RV in the baseline period for the 0.3 mg/kg or 1 mg/kg compound of Formula I groups, and a baseline RV of 0.01±0.01 ml was obtained for the compound of Formula I group at 3 mg/kg. After IV administration of the compound of Formula I 0.3 mg/kg or 1 mg/kg, mean RVs of 0.01±0.01 ml (ns) were obtained, and 0.02±0.02 ml for 3 mg/kg compound of Formula I (ns), indicating no effect on RV with administration of compound of Formula I to normal animals.

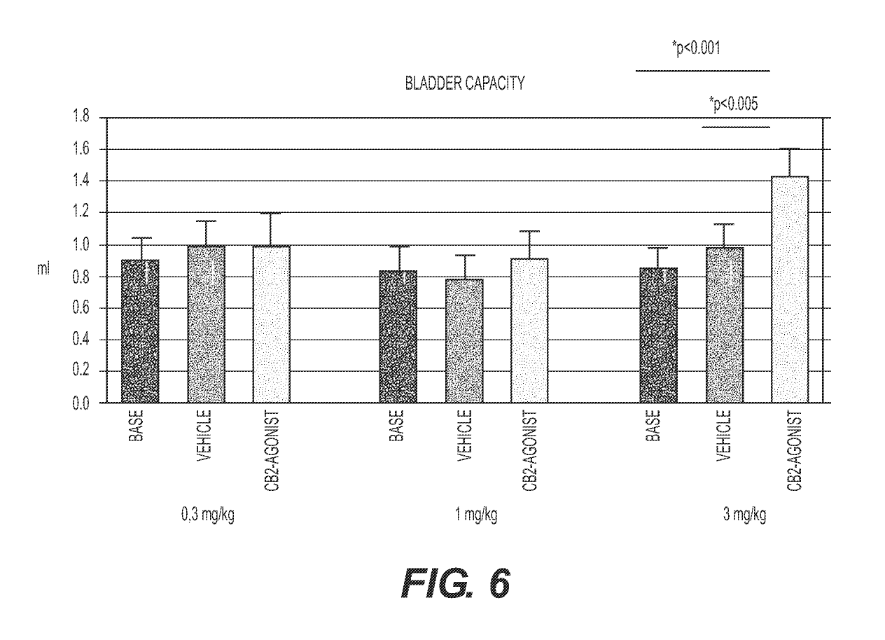

Bladder capacity (“BC”) measured 0.89±0.15 ml (0.3 mg/kg compound of Formula I), 0.82±0.17 ml (1 mg/kg), and 0.83±0.14 ml (3 mg/kg) during the baseline period. At all doses of IV compound of Formula I, 6 out of 8 rats responded with an increased BC. After administration of 0.3 mg/kg or 1 mg/kg, BC was not significantly increased (0.98±0.21 ml and 0.91±0.17 ml, respectively). For 3 mg/kg compound of Formula I, a significant increase in BC was obtained (1.42±0.18 ml (p<0.001 vs. baseline, p<0.005 vs. vehicle) (FIG. 6.). Vehicle did not affect BC (0.98±0.16 ml, 0.78±0.15 ml, and 0.97±0.16 ml).

The above results show that IV administration of the compound of Formula I in conscious normal rats during continuous cystometries had significant effects on parameters that generally may be considered to reflect sensory functions of the micturition reflex. Specifically, MI was increased (FIG. 2), ThP was increased (FIG. 3), MV was increased (FIG. 5) and BC was increased (FIG. 6). These findings together support that acute administration of the compound of Formula I has inhibitory effects on afferent functions of micturition in the normal rat. As MV was increased concomitantly with the increase in MI, and as RV and MP were not significantly altered at the investigated doses of the compound of Formula I, the acute effect of the drug does not appear to interfere with the efficacy of emptying the bladder. The above results demonstrate that CB-2 receptor-mediated mechanisms are most likely involved in regulation of afferent functions of micturition of the normal rat.

### Example 5

### Cystometry in Rats with Partial Urethral Obstruction (“PUO”)

DO can be a result of altered local signals at the level of the detrusor and functional changes of afferent and efferent regulatory mechanisms in the central nervous system (Andersson K E, Arner A. Urinary bladder contraction and relaxation: physiology and pathophysiology. Physiol Rev. 2004; 84:935; Andersson, K E, Wein, A J. Pharmacology of the lower urinary tract: basis for current and future treatments of urinary incontinence. Pharmacol Rev. 2004; 56:581). OAB in men is often associated with urethral obstruction due to benign prostatic hyperplasia (BPH). The PUO model, in which the urethra is partially obstructed with ligature, shows many of the structural and physiological changes in the bladder wall seen in human BPH (McMurray G, Casey J H, Naylor A M. Animal models in urological disease and sexual dysfunction. Br J Pharmacol. 2006; 147 Suppl:S62; Malmgren A, Sjoren C, Uvelius B, Mattiasson A, Andersson K E, Andersson P O. Cystometrical evaluation of bladder instability in rats with infravesical outflow obstruction. J Urol. 1987; 137:1291; Uvelius B, Persson L, Mattiasson A. Smooth muscle cell hypertrophy and hyperplasia in the rat detrusor after short-time infravesical outflow obstruction. J Urol. 1984; 131:173).

In rats with PUO, an increased outflow resistance causes the bladder to significantly enlarge and to hold more urine, and simultaneous compensatory smooth muscle hypertrophy occurs in order for the bladder to be able to empty. Obstructed rats exhibit an increased micturition interval (“MI”) and increased residual volume (“RV”) as indicators for ineffective voiding (Malmgren A, Sjoren C, Uvelius B, Mattiasson A, Andersson K E, Andersson P O. Cystometrical evaluation of bladder instability in rats with infravesical outflow obstruction. J Urol. 1987; 137:1291). An increased volume inside the bladder may be related to increased stretch of the bladder wall and increased tension of the wall of the bladder due to outflow obstruction. Increased volume has been associated with ischemia and structural changes or damage of components of the bladder, which in turn may be related to LUTS (Andersson K E, Arner A. Urinary bladder contraction and relaxation: physiology and pathophysiology. Physiol Rev. 2004; 84:935). Structural or functional changes have been described for different components of the bladder (e.g. smooth muscle, nerves, and urothelium) and for receptor functions at different levels of the peripheral and central nervous system. Obstruction-related patchy cholinergic denervation of the detrusor, enlarged sensory neurons, and altered integrity of the barrier function of the urothelium has been described, and hypothesized as causes for the bladder overactivity (Andersson K E, Arner A. Urinary bladder contraction and relaxation: physiology and pathophysiology. Physiol Rev. 2004; 84:935; Romih R, Korosec P, Jezernik K, Sedmak B, Trsinar B, Deng F M, Liang F X, Sun T T. Inverse expression of uroplakins and inducible nitric oxide synthase in the urothelium of patients with bladder outlet obstruction. BJU Int. 2003; 91: 507).

Other recent evidence suggests that the patchy denervation of the detrusor smooth muscle seen in DO due to PUO is a common feature in the bladder wall of all patients with DO, since this same abnormality was seen in bladders from both male and female patients with idiopathic DO, which also makes this model relevant to female OAB urothelium of patients with bladder outlet obstruction (Mills I W, Greenland J E, McMurray G, McCoy R, Ho K. M, Noble J. G, Brading A F. Studies of the pathophysiology of idiopathic detrusor instability: the physiological properties of the detrusor smooth muscle and its pattern of innervation. J Urol. 2000; 163:646; Turner W H, Brading A F. Smooth muscle of the bladder in the normal and the diseased state: Pathophysiology, diagnosis and treatment. Pharmacol Ther. 1997; 2:77). PUO is also characterized by the presence of non-voiding contractions (“NVC”) which are recorded as continuous occurrences of increases in intravesical pressure during the bladder filling phase (Malmgren A, Sjoren C, Uvelius B, Mattiasson A, Andersson K E, Andersson P O. Cystometrical evaluation of bladder instability in rats with infravesical outflow obstruction. J Urol. 1987; 137:1291).

The effect of chronic treatment with the compound of Formula I on bladder function was studied by cystometry in conscious rats with partial urethral obstruction (“PUO”) after 2 weeks of treatment with the compound of Formula I. Protocols for animals and bladder catheter implantation were as in Example 4 above.

Partial Urethral Obstruction

The bladder and urethra was exposed via a mid-line incision of the lower abdom. A 19 gauge needle (0.9 mm OD) was placed on the surface of the urethra, and a 3-0 black mono nylon suture was firmly tied over the urethra and the ligation was left in place for the duration of the study. The needle was then removed and the diameter and position of the ligature was then inspected to ensure that a partial urethral obstruction was obtained. The abdominal cavity was then closed in layers with single cross-sutures (5-0 black polyfilament nylon suture). Animals were then housed in individual cages for two weeks before cystometry were performed.

Compound of Formula I Preparation and Administration

The appropriate solution of the compound of Formula I was prepared on the day of the experiment using PBS as the vehicle. A stock solution of 10 mg/ml in PBS was used and the animals were administered daily, weight-controlled doses of 3 mg/kg compound of Formula I or vehicle by intraperitoneal (“IP”) injection over the 2 week treatment period, starting on the day of PUO surgery.

Conscious Cystometry

Cystometry was performed without anesthesia three days after the implantation of bladder catheters in rats with PUO, and after two weeks of daily treatment with the compound of Formula I. The conscious rats were placed in metabolic cages without restraint and the bladder catheters were connected via a T-tube to a pressure transducer (P23 DC; Statham Instruments Inc., Oxnard, Calif.) and a microinjection pump (CMA 100; Carnegie Medicine AB, Solna, Sweden). Micturition volumes were recorded with a fluid collector connected to a force displacement transducer (FT 03 D, Grass Instrument Co., Quincy, Mass.). Room-temperature saline was infused into the bladder continuously at a rate of 10 ml/h. Bladder size increases several-fold in rats subjected to PUO. Therefore, an infusion rate of 20 ml/h was used during cystometry in these animals. Pressures and micturition volumes were recorded continuously with Acq Knowledge 3.8.1 software and a MP100 data acquisition system (Biopac Syst. Inc. Santa Barbara, Calif.) connected to a Grass polygraph (Model 7E, Grass Instrument Co). At the beginning of cystometry, the bladder was emptied via the bladder catheter.

Cystometry Measurements and Data Analysis

The following urodynamic parameters were investigated: micturition pressure (“MP;” maximum bladder pressure during micturition), threshold pressure (“ThP”; bladder pressure at start of detrusor contraction), flow pressure (“FP”; bladder pressure when urethra opens), MI (time between two micturitions), micturition volume (“MV”; volume of the expelled urine), and RV (volume left in bladder after a micturition). At least a 30-minute period with stable and reproducible micturitions was recorded before drug administration and used as baseline value, to be compared with a corresponding stable 30-minute period after drug administration. For multiple comparisons, a one-way analysis of variance for repeated measures (Holm-Sidak) was used. Pairwise and non-pairwise comparisons were made by Student's t-test. All statistical calculations are based on the number of individual animals, and significant differences are accepted when p<0.05.

In the chronic study, the incidence of non-voiding contractions (“NVCs”), i.e., the number of animals in each group (vehicle-treated PUO rats and compound of Formula I-treated PUO rats) that exhibited NVCs divided by the total number of animals in each group was analyzed. As used herein, the term “chronic” means that the animals were dosed once/day for 14 days, prior to taking the cystometry measurements. For specific investigation and analysis of NVCs, the PUO should beneficially be removed approximately 2-3 days prior to the urodynamic investigation. However, removal of the PUO is a significantly more extensive surgical procedure than implantation of a bladder catheter alone, and the intervention of removing the PUO per se may cause a local reaction of the outflow region (highest density of sensory nerves of the lower urinary tract), which, in close temporal relation to urodynamic investigations, may have effects on micturition reflex functions. In the current study, the PUO was kept for the full length of the study to ensure that only two parameters affected micturition in the rats: (1) the PUO; and (2) the compound of Formula I. However, it was still possible to investigate the occurrence of NVCs, which were non-voiding bladder contractions registered as increases in bladder pressure exceeding 5 cm H₂O during the filling phase (the interval between voiding) of micturition. The NVCs as set forth above also had to exhibit a continuous and similar pattern throughout the urodynamic investigation. In addition, the NVCs also had to be specifically separated from movement artefacts (noted during the urodynamic investigation) to ensure that the NVC truly originated as a detrusor-contraction. Statistical evaluation of the rate of NVCs in the groups was performed using the Fisher Exact test. A probability of p<0.05 was accepted as significant.

Rats were excluded from the study in the following case: 3 treated and 2 control rats were not used due to urinary retention or bladder decompensation.

Effect of the Compound of Formula I on Micturition in Rats with PUO.

Rats with PUO for 2 weeks exhibited enlarged bladders. For untreated PUO rats, the mean weight of the bladders was 0.99±0.12 g (n=10) and in PUO rats (n=10) treated daily for 2 weeks with the compound of Formula I at 3 mg/kg, IP, the mean bladder weight was not significantly decreased (0.88±0.13 g).

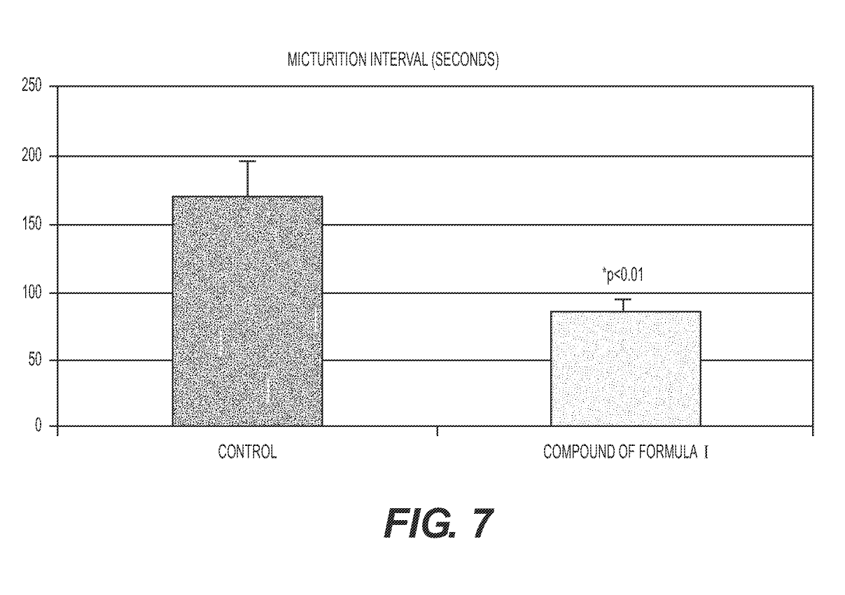

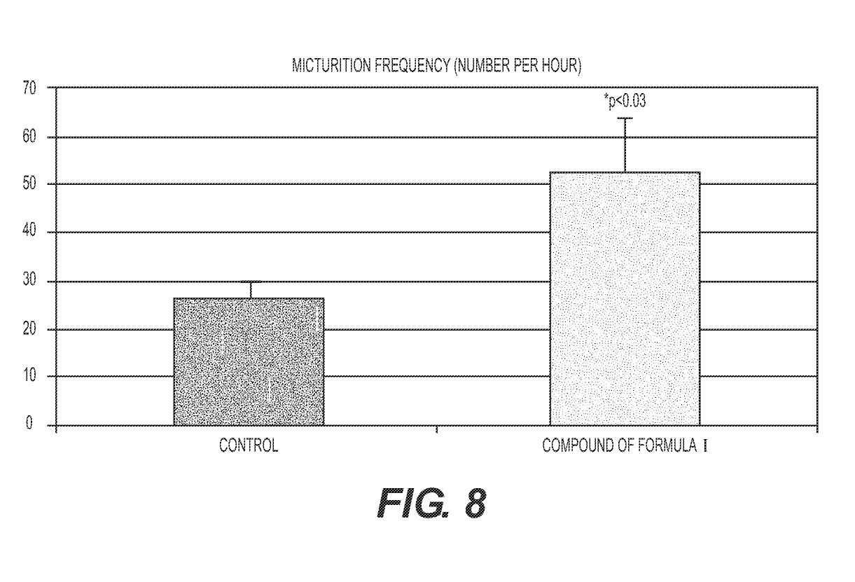

MI in non-treated PUO rats measured 169±28 sec. In PUO rats treated with the compound of Formula I, MI was significantly decreased and amounted to 84±10 sec (p<0.01, FIG. 7). Similarly, micturition frequency (the number of micturitions per hour, FIG. 8) was significantly higher (p<0.03) in compound of Formula I-treated PUO rats (53±11) when compared to non-treated rats with PUO (26±4).

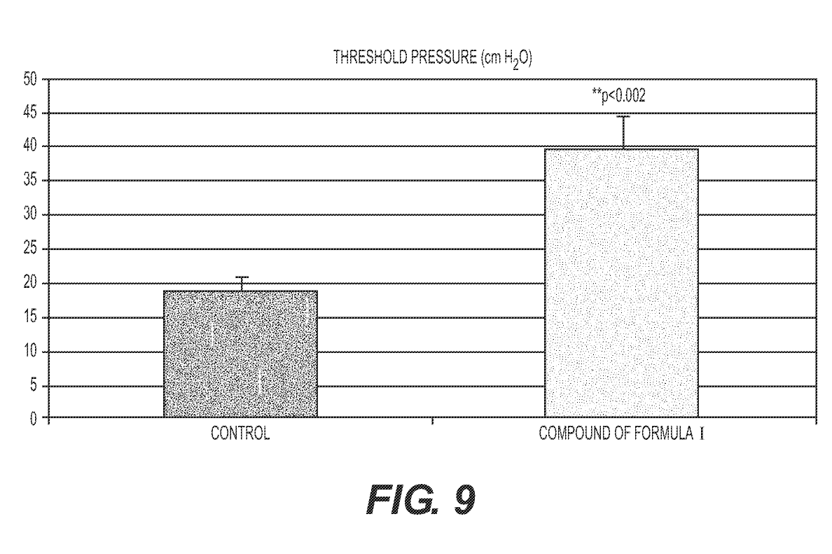

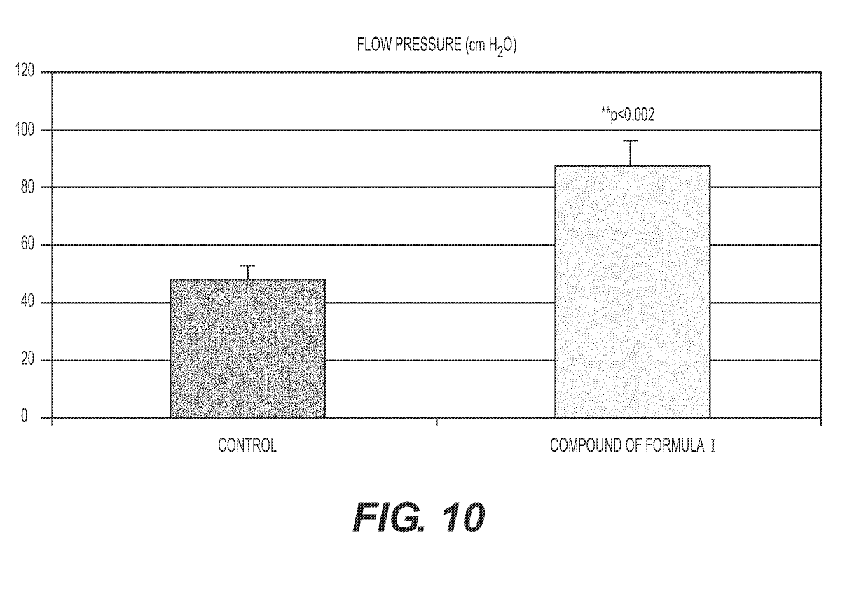

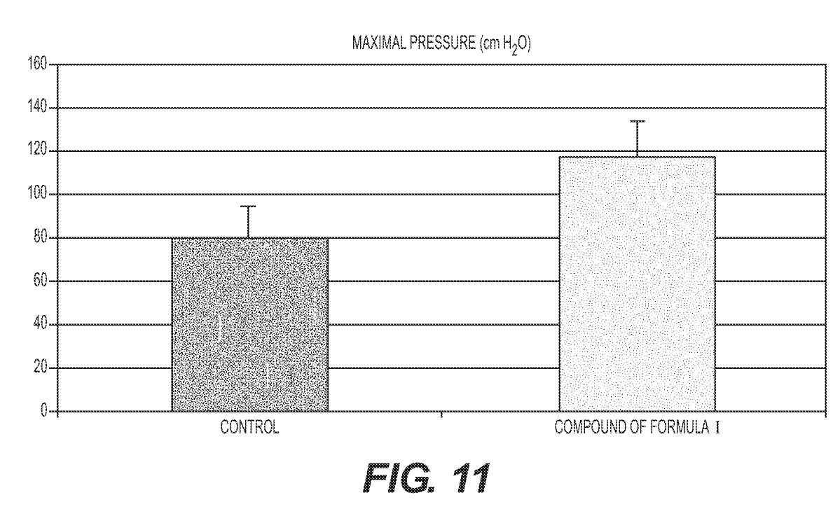

Mean ThP (FIG. 9) of 19.0±2.0 cmH₂O and flow pressure (FP, FIG. 10) of 48.3±5.0 cmH₂O were obtained in non-treated rats with PUO for 2 weeks. Corresponding values for the compound of Formula I-treated rats with PUO were significantly increased with a mean ThP of 39.4±4.9 cmH₂O (p<0.002) and a mean FP of 87.4±8.7 cmH₂O (p<0.002). MP (FIG. 11) in non-treated PUO controls and compound of Formula I-treated rats with PUO was 79.4±14.4 cmH₂O and 116.0±17.4 cmH₂O (ns), respectively.

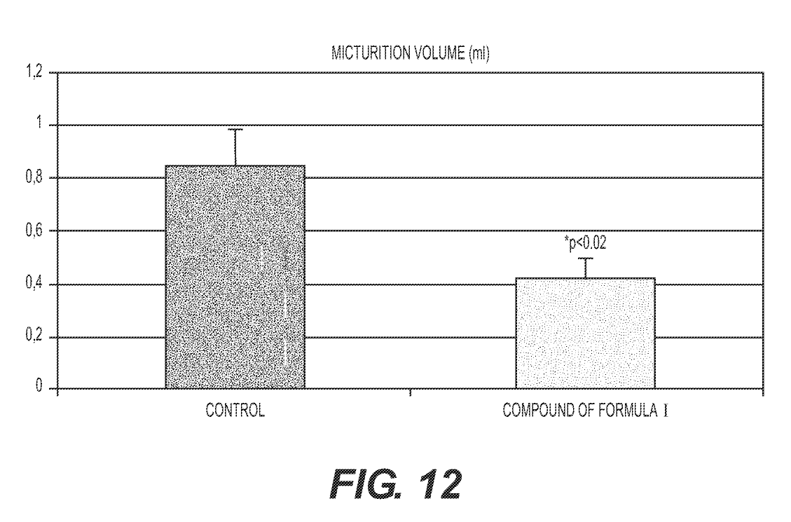

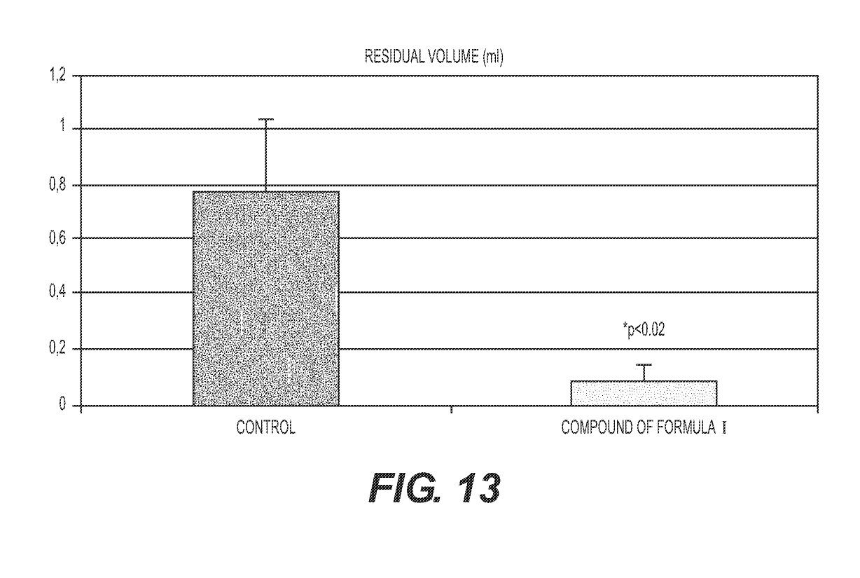

Mean MV (FIG. 12) of 0.84±0.15 ml and residual volume (RV, FIG. 13) of 0.77+0.27 ml were obtained in non-treated rats with PUO. In PUO rats treated with the compound of Formula I, MV and R^{V} significantly decreased to 0.43±0.07 ml (p<0.02) and 0.09±0.05 ml (p<0.02), respectively.

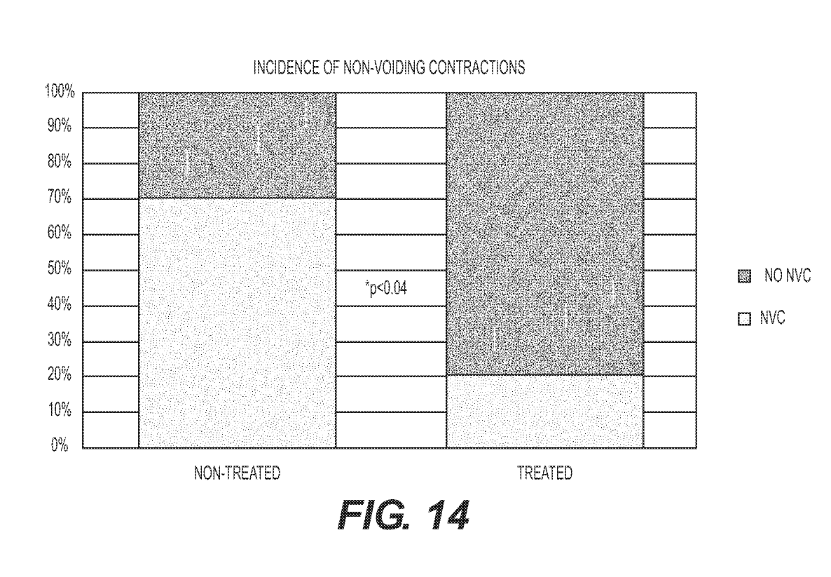

NVCs (FIG. 14) were recorded in 7 out of 10 non-treated rats with PUO. In contrast, only 2 out of 10 PUO rats that were treated with the compound of Formula I exhibited NVCs (p<0.04).

The above results demonstrate that, when comparing non-treated PUO rats to PUO rats treated with the compound of Formula I, MI was decreased and MF was increased for the rats treated with the compound of Formula I (FIGS. 7 and 8). These findings suggest that daily treatment over a two-week period with the compound of Formula I prevented the occurrence of prolonged MI and also counteracted an increase in MV (FIG. 12). Without being bound by theory, the prolonged MI and increase in MV occurred as expected in non-treated rats with PUO. The increase in ThP and FP, which was observed for rats treated with the compound of Formula I (FIGS. 9 and 10), may be related to effects on sensory regulation of the filling phase of the micturition, allowing the bladder to obtain a larger volume before expulsion occurs. Furthermore, an indicator that PUO rats treated with the compound of Formula I exhibited a more efficient and preserved emptying phase during the micturition was the finding that the RV was significantly lower in these rats than for non-treated rats with PUO (FIG. 13).

In the obstructed rat model, DO is a regular finding characterized by NVCs which are recorded as continuous occurrences of increases in intravesical pressure during the filling phase. This was verified by a 70% incidence of NVC in the non-treated PUO control group (FIG. 14). The finding of a reduction of the incidence of NVC in PUO rats treated with the compound of Formula I (20%) (FIG. 14) further supports that the finding that chronic treatment with the compound of Formula I counteracts obstruction-related functional changes of the bladder, and that chronic treatment with the compound of Formula I is effective in the treatment of detrusor overactivity (DO).

The above data show that daily treatment with the compound of Formula I (3 mg/kg for 2 weeks, IP) prevents PUO-induced changes of urodynamic pressure and volume parameters, as well as reduces the incidence of obstruction-related NVC, with CB2-receptor-mediated mechanisms forming the pharmacological basis for such findings.

### Example 6

### In Vitro Contractility Studies in Bladder Strips from Rats with PUO

The effect of chronic treatment with the compound of Formula I was studied in contractility studies with isolated bladder strips from rats with PUO after 2 weeks of treatment with the compound of Formula I.

Functional In Vitro Contractility Experiments in Bladder Strips from Rats with PUO

Bladder strips (2×2×6 mm) were dissected from PUO rat bladders. Silk ligatures were applied at both ends of the preparations which then were mounted on metal prongs in 5 ml aerated (95% O₂ and 5% CO₂) tissue baths (37° C.) containing Krebs solution. Mechanical activity was registered with Grass FT 03C force transducers connected to a Grass Polygraph model 7E. The preparations were stretched to a tension of approximately 10 mN and left to equilibrate for 45 minutes to attain a stable resting tonus. To establish the viability of the preparations and to determine a standard contractile level, preparations were exposed to a 60 mM K⁺ Krebs solution. The effects of cumulative addition of carbachol, a non-selective muscarininc agonist (1 nM-0.1 mM), were studied in strip preparations from vehicle-treated and compound of Formula I-treated rats with PUO.

Transmural activation of nerves was performed with two platinum electrodes, placed in parallel to the sides of the strips in the organ baths. The nerves of the preparations were stimulated by means of a Grass S 48 stimulator, delivering single square-wave pulses at supramaximum voltage with duration of 0.5 ms. The polarity was changed after each pulse by means of a polarity-changing unit. The train duration was 5 seconds and the train interval 120 seconds. Frequency-response relationships were investigated at supramaximum voltage in all preparations stimulated electrically. A preparation was regarded as stable when the amplitude of three consecutive electrically-induced contractions did not differ by more than 5%. All contractile responses were expressed as a percentage of the contraction elicited by 60 mM K⁺ Krebs solution. Simultaneous curve fit analysis was performed using GraphPad Prism v4.0 to determine statistical significance for increased efficacy of the compound of Formula I treatment upon muscle strips. Briefly, dose-response curves were tested comparing a variable slope model vs. a fixed slope (m=1) model. After selection of the best fit model for each curve (fixed slope model), additional analysis using the Global Fit function within Graphpad Prism v4.0 show a statistically significant increase in efficacy conferred by the compound of Formula I over control.

Effect of the Compound of Formula I on Detrusor Contractility in Isolated Bladders from Rats with PUO

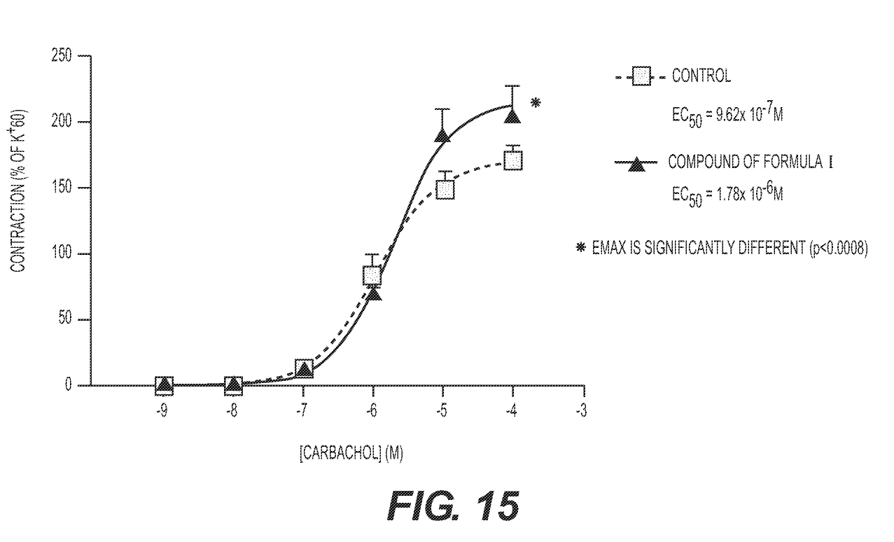

The muscarinic receptor agonist carbachol (1 nM-0.1 mM) produced concentration-dependent contractions of isolated full-wall (intact urothelium and detrusor layers) bladder preparations from all rats after PUO for 2 weeks (FIG. 15). When comparing carbachol-induced detrusor contractions in bladder strips from vehicle-treated PUO rats and from rats with PUO treated with the compound of Formula I for 2 weeks, no differences were observed at carbachol concentrations ranging from 1 nM to 1 μM. However, a significant increase in the maximal response was observed in bladder strips from the rats with PUO treated with the compound of Formula I vs. those from vehicle-treated PUO rats, as revealed by simultaneous curve-fitting analysis of the two dose-response curves (p<0.0008).

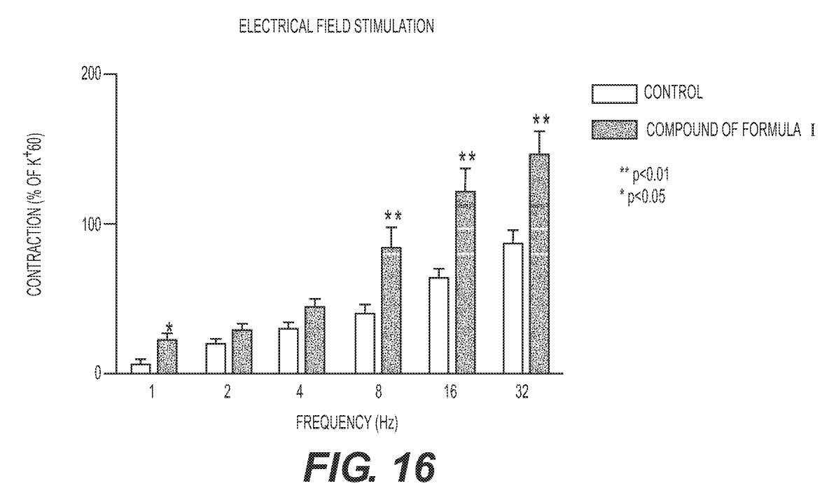

Transmural activation of nerves yielded frequency-dependent contractions in all preparations from PUO rats (FIG. 16). Although significantly larger contractions were noted at 1 Hz in bladders from the compound of Formula I-treated PUO rats compared to untreated PUO rats, even greater differences were observed at 8 Hz (medium high frequency) and at 16 and 32 Hz (high frequencies). At 8 Hz, activation of nerves produced contractions amounting to 40±6% and 83±14% (p<0.01) of 60 mM K⁺-responses in preparations from non-treated PUO rats from PUO rats treated with the compound of Formula I, respectively. Contractions measuring 63±7% (untreated PUO rats) and 120±16% (compound of Formula I-treated PUO rats, p<0.005) were obtained at 16 Hz, and 86±10% (untreated PUO rats) and 146±16% (compound of Formula I-treated PUO rats, p<0.006) were recorded at 32 Hz.

The above results demonstrate that in functional in vitro experiments using isolated detrusor preparations from rats, there was an improved postjunctional responsiveness to muscarinic receptor-mediated contractions, evidenced by the increased maximal response (Emax) in the strips from obstructed rats that had been treated by the compound of Formula I vs. the strips from obstructed rats that had been treated with vehicle alone. The postjunctional cholinergic receptor system is part of the efferent nervous system that controls the voiding contraction during the emptying phase of the micturition response. Thus, the carbachol dose-response data (FIG. 15) may be suggestive of a better status of the smooth muscle component of the detrusor wall, and, in particular, an improvement in the status of the smooth muscle component of the voiding contraction. In addition, autonomic motor nerve functions were significantly better in detrusor preparations from rats treated with the compound of Formula I (FIG. 16), and it may be that the patchy denervation associated with outflow obstruction can be prevented by chronic treatment with the compound of Formula I.

### Example 7

### Cystometry in the Cyclophosphamide-Induced Model of Interstitial Cystitis/Painful Bladder Syndrome (“IC/PBS”)

This study involved the determination of the acute effect of the compound of Formula I on bladder function in conscious rats in the cyclophosphamide-induced model of interstitial cystitis/painful bladder syndrome (“IC/PBS”) using cystometry. The clinical use of cyclophosphamide (“CYP”), a chemotherapy drug that is given as a treatment for many types of cancer, can cause inflammation of the bladder lining, inducing acute hemorrhagic cystitis or chronic cystitis, and result in side effects in the bladder that can be seen in patients with IC/PBS. (Levine L A, Richie J P. Urological complications of cyclophosphamide. J Urol. 1989; 141:1063; Stillwell T J, Benson R C. Cyclophosphamide-induced cystitis. A review of 100 patients. Cancer. 1988; 61:451). The cystitis is not caused by CYP itself, but to acrolein, a toxic CYP metabolite produced by microsomal metabolism that is eliminated by the bladder (Levine L A, Richie J P. Urological complications of cyclophosphamide. J Urol. 1989; 141:1063). In rats, IP administration of CYP induces bladder inflammation and disrupts the urothelial lining, increasing afferent nerve sensitivity and leading to bladder overactivity. Bladder weight is increased due to the inflammation, and bladder capacity is decreased (Corrow K A, Vizzard M A. Phosphorylation of extracellular signal-regulated kinases in urinary bladder in rats with cyclophosphamide-induced cystitis. Am J Physiol. 2007; 293:R125; Lecci A, Birder L A, Meini S, Catalioto R-M, Tramontana M, Giuliani S, Criscuoli M, Maggi C A. Pharmacological evaluation of the role of cyclooxygenase isoenzymes on the micturition reflex following experimental cystitis in rats. Br J Pharmacol. 2000; 130:331).

Animals

Male Sprague Dawley rats, weighing 250 g, were maintained under standard laboratory conditions with a 12:12 light/dark cycle and free access to food pellets and water. For surgical procedures, the rats were anesthetized with IP administration of Equinthensin solution (3 ml/kg).

Bladder and Intravenous (IV) Catheter Implantation

The bladder was exposed via a mid-line incision of the lower abdomen, and the bladder was freed from adhering tissues and emptied. A saline-filled polyethylene catheter (0.58 mm ID and 0.96 mm OD) was implanted in the bladder via an incision at the bladder dome and sutured in place with silk thread. For IV compound administration, a similar PE catheter filled with saline containing sodium heparin (40 IU/ml) was inserted into a jugular vein. The cannulas were exteriorized through a subcutaneous tunnel in the retroscapular area and are connected with a plastic adaptor in order to avoid removal by the animal.

Cyclophosphamide and Compound of Formula I Preparation and Administration

Cyclophosphamide was dissolved in distilled water and injected at 150 mg/kg, intraperitoneal (“IP”), to induce bladder cystitis 24 hr prior to cystometry. The compound of Formula I was initially dissolved in a vehicle containing a co-solvent mixture of Cremophor EL® (70%, BASF Corporation, Florham Park, N.J.) and ethanol (30%) as a 20-fold concentrated stock solution at 40 mg/ml. Prior to use, the 20× compound stock solution or vehicle was diluted 1:20 dilution (v:v) with saline to give a 2 mg/ml final concentration for dosing. The compound of Formula I or vehicle was administered IP in a volume of 5 ml/kg for a final dose compound of Formula I of 10 mg/kg, IP.

Conscious Cystometry

Rats were placed in transparent restraining cages to allow measurement of micturition volume (MV). After a 20 min stabilization period, the free tip of the bladder cannula was connected to a pressure transducer for the continuous measurement of bladder pressure, and a peristaltic pump for the continuous infusion of saline into the bladder at a constant filling rate of 0.05 ml/min.

Cystometry Measurements and Analysis

Bladder capacity (“BC”) and micturition pressure (“MP”) were the urodynamic parameters evaluated from the cystometrogram. BC (ml) was defined as the bladder infused volume at the time when detrusor contraction was followed by micturition. MP (mm Hg) was defined as the maximal intravesical pressure induced by the contraction of the detrusor during micturition. Micturition volume (“MV”), defined as the volume of urine expelled during each individual micturition cycle, was also measured by means of a force displacement transducer connected to a recording polygraph that measured the urine collected in a small reservoir placed under the cage. These recordings were used only for inspection of effective voiding contractions.

Basal urodynamic parameters were evaluated as mean values from representative cystometrograms that were recorded in a 30-60 minute time period prior to cyclophosphamide injection (Ohr basal values, obtained before CYP treatment). Following the basal cystometry period, CYP was administered by IP injection 24 hr before the next cystometry period. BC and MP were then acquired for 1 hr in the CYP-treated rats (24 hr values), followed by vehicle or compound of Formula I treatment IP. After vehicle or compound of Formula I administration, the time course of the BC and MP were then followed for an additional 5 hr (25-29 hr) in the CYP-treated rats.

Effect of the Compound of Formula I on Micturition in Conscious Rats with CYP-Induced IC/PBS

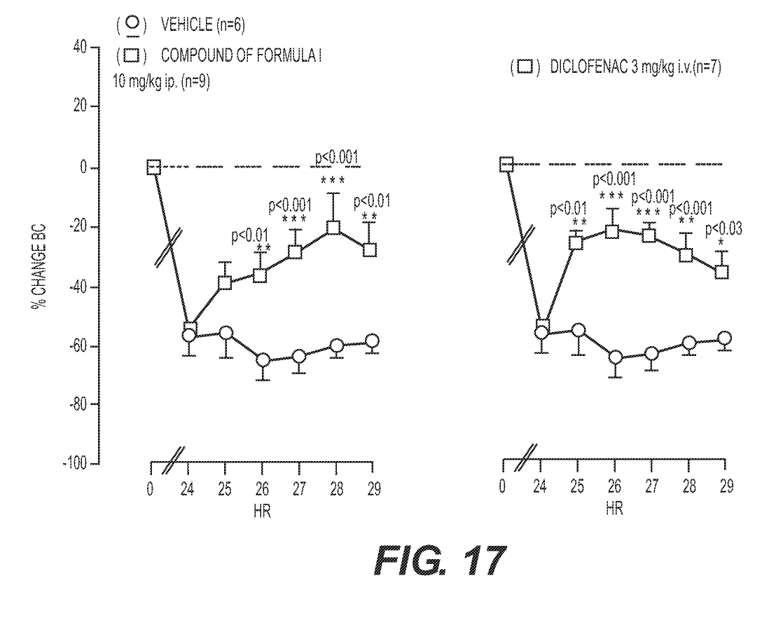

Cyclophosphamide administration decreased BC from 0.80±0.05 ml to 0.36±0.03 ml (a 55% reduction compared to pre-CYP treatment levels) at 24 hr after CYP injection (FIG. 17). BC remained at approximately that same level in the vehicle-treated CYP rats for the next 5 hours during cystometry. Treatment with the compound of Formula I (10 mg/kg, IP) significantly reversed the decreased BC in the CYP-treated rats by 2 hr post treatment (0.57±0.06 ml), to a 29% reduction (26 hr time point). BC remained significantly greater in the compound of Formula I-treated CYP rats for the rest of the 5 hr monitoring period after treatment with the compound of Formula I, and increased to as much as 0.63±0.09 ml (21% reduction, 28 hr time point) at 4 h after administration of the compound of Formula I.

The above results indicate that the compound of Formula I is effective in attenuating the irritative damage induced by cyclophosphamide treatment, as reflected by the improved ability of the bladder to retain urine.

### Example 8

### Use of a CB2-Receptor-Selective Agonist in Combination with a Muscarinic Receptor Antagonist in the PUO Model

Darifenacin is an M3-receptor-selective muscarinic antagonist with limited blood-brain barrier penetration (Hiroyasu Hirose, Ikuo Aoki, Toshifumi Kimura, Toru Fujikawa, Tomoshige Numazawa, Kaori Sasaki, Akio Sato, Takuro Hasegawa, Masaru Nishikibe, Morihiro Mitsuya, Norikazu Ohtake, Toshiaki Mase, and Kazuhito Noguchi. Pharmacological Properties of (2R)-N-[1-(6-Aminopyridin-2-ylmethyl)piperidin-4-yl]-2-[(1R)-3,3-difluorocyclopentyl]-2-hydroxy-2-phenylacetamide: A Novel Muscarinic Antagonist with M2-Sparing Antagonistic Activity. J Pharmacol Exp Ther 297 (2):790-797, 2001; Shuji Maruyama, Hideo Tsukada, Shingo Nishiyama, Takeharu Kakiuchi, Dai Fukumoto, Naoto Oku, and Shizuo Yamada. In Vivo Quantitative Autoradiographic Analysis of Brain Muscarinic Receptor Occupancy by Antimuscarinic Agents for Overactive Bladder Treatment. J Pharmacol Exp Ther 325 (3):774-781, 2008) currently marketed for the treatment of overactive bladder. Darifenacin is (S)-2-{1-[2-(2,3-dihydrobenzofuran-5-yl)ethyl]-3-pyrrolidinyl}-2,2-diphenyl-acetamide, disclosed in European Patent No 0388054, Examples 1B and 8, and is referred to therein as 3-(S)-(−)-(1-carbamoyl-1,1-diphenylmethyl)-1-[2-(2,3-dihydro-benzofuran-5-yl)ethyl]pyrrolidine. Darifenacin and pharmaceutical formulations comprising darifenacin are disclosed in U.S. Pat. No. 6,106,864.

Darifenacin will be administered intravenously (“IV”) at doses of 0.01, 0.03, 0.1, and 0.3 mg/kg alone or in combination with different doses of a CB2-receptor-selective agonist in the partial urethral obstruction model as described in Example 5 to look for synergy in the improvement in the reduction of nonvoiding contractions and the improvement in bladder function, over that seen with the dose of either agent alone. Alternatively, darifenacin will be administered orally at doses of 0.05, 0.1, 0.3 and 0.6 mg/kg alone and in combination with different doses of a CB2-receptor-selective agonist in the partial urethral obstruction model as described in Example 5 to look for synergy in the improvement in the reduction of nonvoiding contractions and the improvement in bladder function, over that seen with the dose of either agent alone. The different doses of darifenacin and the CB2-receptor-selective agonist will be administered separately, within 3 minutes of each other to the same animal, once daily for 14 days as described in Example 5, and the response would be measured by conscious cystometry after two weeks of daily treatment with the combination as described in Example 5.

### Example 9

### Use of a CB2-Receptor-Selective Agonist in Combination with a Muscarinic Receptor Antagonist in Humans

Therapeutic doses of darifenacin in humans include 7.5 mg and 15 mg. A clinical trial will be run that uses 7.5 mg of darifenacin with 1-2 different doses of a CB2-receptor-selective agonist. These will be administered in a single formulation in a multi-arm study as follows: placebo, 7.5 mg darifenacin alone, 1-2 different doses of a CB2-receptor-selective agonist alone, and a combination of 7.5 mg darifenacin+dose 1 of CB2-receptor-selective agonist, and/or a combination of 7.5 mg darifenacin+dose 2 of CB2-receptor-selective agonist. Improvements in symptom relief over that seen with placebo and with darifenacin alone, as assessed by patient dairies, will be analyzed. Improvements in bladder function over that seen with placebo and with darifenacin alone, as assessed by urodynamic measurement, will be analyzed.

While this invention has been particularly shown and described with references to embodiments thereof, it will be understood by those skilled in the art that various changes in form and detail may be made therein without departing from the scope of the invention encompassed by the claims. Such various changes that will be understood by those skilled in the art as covered within the scope of the invention include, in particular, methods directed to administering CB2 receptor-selective agonists other than the classes of cannabinoids described herein, including compounds other than the compounds of Formulas I, II, III, and IV.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed as new and desired to be protected by Letters Patent of the United States is:

1. A method for treating or preventing lower urinary tract dysfunction in a mammal, which comprises administering to said mammal a therapeutically effective amount of a CB2-receptor-selective agonist.

2. The method of claim 1, wherein said CB2-receptor-selective agonist is peripherally selective.

3. The method of claim 2, wherein said CB2-receptor-selective agonist is a compound of Formula I: 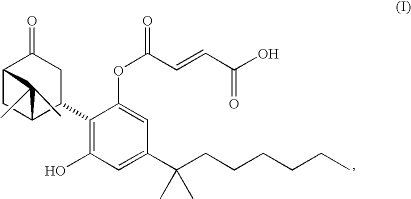
or a pharmaceutically acceptable salt, ester or solvate thereof.

4. The method of claim 1, wherein said CB2-receptor-selective agonist is a compound of Formula II: 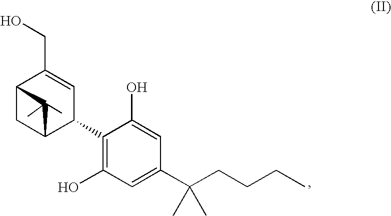
or a pharmaceutically acceptable salt, ester or solvate thereof.

5. The method of claim 1, wherein said CB2-receptor-selective agonist is a compound of Formula III: 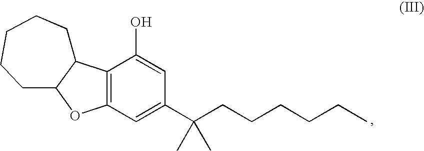
or a pharmaceutically acceptable salt, ester or solvate thereof.

6. The method of claim 1, wherein said CB2-receptor-selective agonist is a compound of Formula IV: 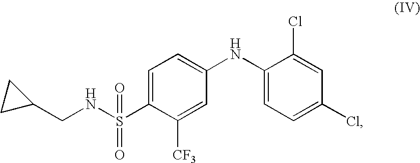
or a pharmaceutically acceptable salt, ester or solvate thereof.

7. A method for treating or preventing overactive bladder, lower urinary tract symptoms or detrusor overactivity in a mammal, which comprises administering to said mammal a therapeutically effective amount of a CB2-receptor-selective agonist.

8. A method for preserving an ability to empty the bladder in a mammal, which comprises administering to said mammal a therapeutically effective amount of a CB2-receptor-selective agonist.

9. A method for preventing deterioration of or protecting bladder contractility in a mammal, which comprises administering to said mammal a therapeutically effective amount of a CB2-receptor-selective agonist.

10. A method for improving bladder function in a mammal, which comprises administering to said mammal a compound of Formula I below: 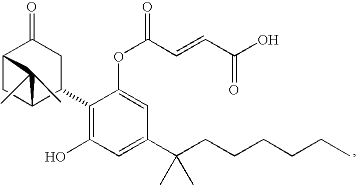
or a pharmaceutically acceptable salt, ester or solvate thereof.

11. The method of claim 3, wherein said CB2-receptor-selective agonist is administered orally in a total daily dose of about 10 mg to about 300 mg.
